(19) United States
(12) Patent Application Publication
(10) Pub. No.: US 2022/0220441 A1
(43) Pub. Date: Jul. 14, 2022

(54) RAPID AND DETERMINISTIC GENERATION OF MICROGLIA FROM HUMAN PLURIPOTENT STEM CELLS

(71) Applicant: Westfälische Wilhelms-Universität Münster, Munster (DE)

(72) Inventors: Matthias Pawlowski, Munster (DE); Anna Martina Speicher, Osnabrock (DE)

(21) Appl. No.: 17/613,927

(22) PCT Filed: May 27, 2020
(86) PCT No.: PCT/EP2020/064649

§ 371 Date: Nov. 23, 2021

(30) Foreign Application Priority Data
May 27, 2019 (EP) 19176722.7

(51) Int. Cl. C12N 5/079; C07K 14/53; C07K 14/54

(52) U.S. Cl. CPC C12N 5/0622; C07K 14/53; C07K 14/54; C12N 2506/45; C12N 2501/22; C12N 2501/2334; C12N 2502/08; C12N 2830/003

(57) ABSTRACT

The present invention relates to a method for the production of microglia from stem cells comprising the steps of a) targeted insertion of a nucleotide sequence encoding a transcriptional regulator protein into a first genomic safe harbour site; and b) targeted insertion of the coding sequence of the transcription factor PU.1 (SEQ ID NO: 1) into a second genomic safe harbour site, wherein the gene is operably linked to an inducible promoter, which is regulated by the transcriptional regulator protein; expression of PU.1 (SEQ ID NO: 2); and culturing the stem cells received from steps a) and b) with exposure to at least one growth factor or small molecule that mimics signaling during at least one stage of embryonic development of microglia or adult microglia proliferation, differentiation or polarization. Further, the present invention relates to the microglia obtained by the methods of the present invention and various uses thereof.

### FIELD OF THE INVENTION

The present invention relates to a method for the production of microglia from stem cells comprising the steps of targeted insertion of a nucleotide sequence encoding a transcriptional regulator protein into a first genomic safe harbour site; and targeted insertion of the coding sequence of the transcription factor PU.1 into a second genomic safe harbour site, wherein the gene is operably linked to an inducible promoter, which is regulated by the transcriptional regulator protein; expression of PU.1; and culturing the stem cells received from steps a) and b) with exposure to at least one growth factor or small molecule that recapitulates signaling during at least one stage of embryonic development of microglia or adult microglia proliferation, differentiation or polarization. Further, the present invention relates to the microglia obtained by the methods of the present invention and various uses thereof.

### BACKGROUND OF THE INVENTION

Microglia are the resident immune cells in the central nervous system (CNS) [Schafer et al., 2015]. They originate from early yolk sac macrophages that arise during the first wave of primitive haematopoiesis in early embryonic development. Primitive yolk sac macrophages spread through the blood stream as soon as the circulatory system is established to populate the developing CNS. In contrast to tissue-resident macrophages in other organs, microglia are not replaced by foetal monocytes during later stages of embryonic development [McGrath et al., 303; Ginhoux et al., 2010; Gomez Perdiguero et al., 2015]. After establishing the microglial population during early embryonic development, microglia are self-maintained throughout life by local proliferation, not replaced by bone-marrow-derived cells [Réu et al., 2017]. Microglia are uniformly distributed throughout the brain and spinal cord and play crucial roles in the development, maintenance, plasticity and defence of the CNS [Schafer et al., 2015]. In the healthy CNS, “resting” homeostatic microglia are highly ramified cells with a small cell body and fine cellular processes. These microglial processes are motile and continuously sampling their environment to scan for signals of internal or external danger (such as invading pathogens or signals generated locally by damaged or dying cells). Detection of such signals leads to microglial activation, which comprises profound changes in microglial morphology, gene expression, and function. Upon activation, microglia retract their processes and revert to an amoeboid-like appearance. They actively migrate to CNS lesions following chemotactic gradients and secrete inflammatory cytokines.

Through a large repertoire of cell surface receptors, including neurotransmitter and cytokine receptors, they communicate with neurons, other glial cells, and peripheral immune system cells [Kettenmann et al., 2011]. In light of their versatile functions and their unique position as representatives of the immune system in the healthy CNS, it is not surprising that microglia have been implicated in the onset and progression of many neurological diseases [Ransohoff et al., 2016].

Most recently, single cell transcriptomic profiling of microglia in mouse models of Alzheimer's disease (AD) and other neurodegenerative diseases including ageing, amyotrophic lateral sclerosis or tauopathy-related frontotemporal lobar degeneration (FTLD-tau), have revealed a pro-inflammatory transcriptomic signature in a small subset of microglia termed microglial neurodegenerative phenotype (MGnD) [Krasemann et al., 2017] or disease-associated microglia (DAM) [Keren-Saul et al., 2017]. The microglial switch from a homeostatic towards a disease-associated phenotype is thought to occur in response to altered brain homeostasis in neurodegeneration and is dependent on unique temporally and spatially controlled transcriptional programmes [Krasemann et al., 2017; Keren-Shaul et al., 2017; Butovsky et al., 1998]. In most cases, it remains unclear whether these cells have a protective or disease-inducing/propagating function. Access to human microglia in vitro and in vivo, in health and disease, would facilitate the identification of factors associated with both their beneficial and detrimental functions and the development of strategies to restore the homeostatic microglial signature or to induce the DAM microglial signature. This could allow us to target microglia for the treatment of neurodegenerative diseases.

The isolation or in vitro derivation of many human cell types remains challenging and inefficient. Especially cells of the human CNS, including microglia, are particularly difficult to obtain. In the past, low-efficient isolation from neurosurgical specimen or post-mortem brain tissue represented the only access route. Human pluripotent stem cells (hPSCs) represent an unlimited and renewable source from which, in theory, all cell types of the human organism can be produced [Thomson et al., 1998]. The ground-breaking discovery that human skin fibroblasts can be readily converted into human induced pluripotent stem cells (hiPSCs) that exhibit the same properties as embryonic stem cells, allows the generation of autologous and bespoke cell types for applications in regenerative medicine. For several key applications including disease modelling, drug discovery, and cell transplantation, large-scale manufacture of mature human cell types from hPSCs is required. Recently, the first hPSC-differentiation protocol for the generation of microglia was published. It was based on the initial formation of embryoid-bodies (EBs) cultured for several months in the same “neuroglial differentiation medium, the component concentrations of which were adjusted to match those of human cerebrospinal fluid” supplemented with interleukin (IL)-34 and colony-stimulating factor 1 (CSF-1) [Muffat et al., 2016]. This seminal publication provided an elaborate media composition for final maturation and maintenance of human microglia. However, the long duration of the protocol, ill-defined initial steps of differentiation (i.e. EB-based, intermediate steps hardly following embryonic rationales), and the need for several mechanical manipulation steps for cell purification are likely to prohibit the widespread application of this protocol. Subsequently, several other groups demonstrated the generation of microglia-like cells from hPSCs by similar, yet different classical differentiation approaches [Abud et al., 2017; Takata et al., 2017; Haenseler et al., 2017; Pandya et al., 2017; Douvaras et al., 2017]. Nonetheless, the in vitro derivation of specific human cell types, including microglia, in a quantity and purity that is required for downstream applications remains challenging, and alternative methods are currently sought [Cohen et al., 2011]. A more recent manufacturing strategy compared to classical differentiation is direct cellular reprogramming [Ladewig et al., 2013]. It refers to the direct conversion of any cell type (typically skin fibroblasts) into another without progression through a pluripotent intermediate. Although providing a quick route for cell production from easily accessible cell types, the yield and purity of the desired cell populations remain low and insufficient [Zhang et al., 2013]. Recently, a third route, termed “forward programming”, was proposed for the manufacture of mature human cell types with unprecedented speed and efficiency [Zhang et al., 2013].

Forward programming, as a method of directly converting pluripotent stem cells, including hPSCs, to mature cell types has been recognised as a powerful strategy for the derivation of human cells. It involves the forced expression of key lineage transcription factors (or non-coding RNAs, including IncRNA and microRNA), in order to convert the stem cell into a particular mature cell type. Currently available forward programming protocols are largely based on lentiviral transduction of cells, which results in variegated expression or complete silencing of randomly inserted inducible cassettes. This results in the need for additional purification steps in order to isolate a sub-population expressing the required transcription factors. Thus, further refinements of these methods are clearly required.

Any refinements to the stated methods must ensure that stable transcription of the genetic material contained within the inducible cassette, such as a transgene, is resistant to silencing and other negative integration site-related influences. Silencing may be caused by multiple epigenetic mechanisms, including DNA methylation or histone modifications. With prior art methods based on lentiviral transduction, the cells obtained are a heterogeneous population with the transgene expressed fully, partially or silenced. Clearly, this is not desirable for many applications. Viral vectors demonstrate a tendency to integrate their genetic material into transcriptionally active areas of the genome, thus increasing the potential for oncogenic events due to insertional mutagenesis. For many applications, it is desirable to control the transcription of inserted genetic material in a cell, such that an inducible cassette may be turned on as required and transcribed at particular levels, including high levels. This cannot be achieved if the insertion of the inducible cassette is random in the genome.

The problem, of microglia being both involved in several serious diseases and entangled into the brain tissue in a way that their isolation from living tissue remains elusive, has been addressed in several publications. To overcome this problem, human stem cells are used to generate microglia or microglia-like cells for example through defined culturing conditions [Muffat et al., 2016] or co-culturing with stem cell derived neurons [Haenseler et al., 2017; Takata et al., 2017]. These methods rely only on the exposure to growth factors and cytokines to differentiate stem cells into microglia.

Further the need for this special cell type is huge as they play an important role in virtually all diseases of the central nervous system, including neurodegenerative diseases, neuroinflammatory or autoimmune diseases, auto-antibody-mediated encephalitis or infectious diseases, neurovascular diseases, stroke, traumatic brain injuries and cancer, yet the precise mechanisms underlying their role in different diseases remain unclear. Prior art coincides, stem cell-derived microglia are indeed recapitulating the original patients disease-phenotype [Muffat et al., 2016; Abud et al., 2017; Takata et al., 2017]. With this knowledge, the enormous scientific gap of microglia involvement in certain diseases can be overcome by generating microglia from stem cells. However the classical protocols to differentiate stem cells are very time-consuming and the results are not convincing.

The inventors of the present invention have thus developed a quick method for generating microglia from stem cells by using a stable introduction of an inducible cassette into the genome of a stem cell, whilst being able to control the transcription of that inducible cassette and thereby the inserted transcription factors. The potential of these transcription factors to function as reprogramming factors for the generation of microglia was not known before and represents the unique knowledge of the inventors. This enables them to create a pure microglia population expressing all the surface markers and RNA observed in natural microglia populations. Moreover this method can be used to differentiate microglia from human iPS cells of neurodegenerative disease patients and thus enables to analyse a cell population that otherwise remains completely inert to medical examinations. Accordingly, there is a strong need for manufacture of mature human microglia from easily accessible sources. The technical problem underlying the present application is thus to comply with these needs. The technical problem is solved by providing the embodiments reflected in the claims, described in the description and illustrated in the examples and figures given below.

### SUMMARY OF THE INVENTION

The inventors of the present invention have developed a method for the production of microglia from stem cells.

The present invention relates to a method for the production of microglia from stem cells, comprising the steps of a) targeted insertion of a nucleotide sequence encoding a transcriptional regulator protein into a first genomic safe harbour site; and b) targeted insertion of the coding sequence of the transcription factor PU.1 (SEQ ID NO: 1) into a second genomic safe harbour site, wherein the gene is operably linked to an inducible promoter, which is regulated by the transcriptional regulator protein; expression of PU.1 (SEQ ID NO: 2); and c) culturing the stem cells received from steps a) and b) with exposure to at least one growth factor or small molecule that recapitulates signaling during at least one stage of embryonic development of microglia or adult microglia proliferation, differentiation or polarization.

In one embodiment of the method of the present invention, the at least one growth factor or small molecule is selected from the group consisting of Activin A (SEQ ID NO: 7), BMP4 (SEQ ID NO: 8), FGF (SEQ ID NO: 9), VEGF-A (SEQ ID NO: 10), LY294002, CHIR99021, SCF (SEQ ID NO: 11), IL-3 (SEQ ID NO: 12), IL-6 (SEQ ID NO: 13), CSF1 (SEQ ID NO: 14), IL-34 (SEQ ID NO: 15), CSF2 (SEQ ID NO: 16), CD200 (SEQ ID NO: 17), CX3CL1 (SEQ ID NO: 18), TGFβ1 (SEQ ID NO: 19), and IDE1.

In a further embodiment of the method of the present invention, the at least one growth factor is CSF1 (SEQ ID NO: 14) or IL-34 (SEQ ID NO: 15).

In an additional embodiment of the method of the present invention, the at least one small molecule is CHIR99021, LY294002 or IDE1.

In another embodiment of the method of the present invention, the first and the second genomic safe harbour sites are different.

In a further embodiment of the method of the present invention, the method further comprises insertion of the coding sequence of the gene of the transcription factor CEBPB (SEQ ID NO: 3) and expression thereof.

In another embodiment of the method of the present invention, the method further comprises insertion of the coding sequence of the gene of the transcription factor RUNX1 (SEQ ID NO: 4) and expression thereof.

In a further embodiment of the method of the present invention, the method further comprises insertion of the coding sequence of the gene of the transcription factor IRF8 (SEQ ID NO: 5) and expression thereof.

In another embodiment of the method of the present invention, the method further comprises insertion of the coding sequence of the gene of the transcription factor SALL1 (SEQ ID NO: 6) and expression thereof.

In an additional embodiment of the method of the present invention, the transcriptional regulator protein is the reverse tetracycline transactivator (rtTA) (SEQ ID NO: 20) and the activity thereof is controlled by doxycycline or tetracycline.

In another embodiment of the method of the present invention, the inducible promoter includes a Tet Responsive Element (TRE) (SEQ ID NO: 21).

In a further embodiment of the method of the present invention, said first and said second genomic safe harbour sites are selected from the group consisting of the hROSA26 locus (SEQ ID NO: 22), the AAVS1 locus (SEQ ID NO: 23), the CLYBL gene (SEQ ID NO: 24), the CCR5 gene (SEQ ID NO: 25), the HPRT gene (SEQ ID NO: 26) or genes with the site ID 325 on chromosome 8 (SEQ ID NO: 27), site ID 227 on chromosome 1 (SEQ ID NO: 28), site ID 229 on chromosome 2 (SEQ ID NO: 29), site ID 255 on chromosome 5 (SEQ ID NO: 30), site ID 259 on chromosome 14 (SEQ ID NO: 31), site ID 263 on chromosome X (SEQ ID NO: 32), site ID 303 on chromosome 2 (SEQ ID NO: 33), site ID 231 on chromosome 4 (SEQ ID NO: 34), site ID 315 on chromosome 5 (SEQ ID NO: 35), site ID 307 on chromosome 16 (SEQ ID NO: 36), site ID 285 on chromosome 6 (SEQ ID NO: 37), site ID 233 on chromosome 6 (SEQ ID NO: 38), site ID 311 on chromosome 134 (SEQ ID NO: 39), site ID 301 on chromosome 7 (SEQ ID NO: 40), site ID 293 on chromosome 8 (SEQ ID NO: 41), site ID 319 on chromosome 11 (SEQ ID NO: 42), site ID 329 on chromosome 12 (SEQ ID NO: 43), site ID 313 on chromosome X (SEQ ID NO: 44).

In another embodiment of the method of the present invention, said stem cell is a pluripotent stem cell, an induced pluripotent stem cell (iPSC), a neural progenitor cell, hematopoietic stem cell or an embryonic stem cell (ESC).

In a further embodiment of the method of the present invention, said stem cell is a human or a mouse stem cell.

The present invention also relates to a microglia cell obtained by any of the methods according to the present invention, preferably wherein the microglia expresses at least one microglia surface protein selected from the group consisting of ITGAM (CD11B) (SEQ ID NO: 45), ITGAX (CD11C) (SEQ ID NO: 46), CD14 (SEQ ID NO: 47), CD16 (SEQ ID NO: 48), ENTPD1 (CD39) (SEQ ID NO: 49), PTPRC (CD45) (SEQ ID NO: 50), CD68 (SEQ ID NO: 51), CSF1R (CD115) (SEQ ID NO: 52), CD163 (SEQ ID NO: 53), CX3CR1 (SEQ ID NO: 54), TREM2 (SEQ ID NO: 55), P2RY12 (SEQ ID NO: 56), TMEM119 (SEQ ID NO: 57), and HLA-DR (SEQ ID NO: 58).

In a further embodiment of the present invention, the microglia cell is for use in therapy.

Further, the present invention is directed to the use of such a microglia cell according to the present invention for in vitro diagnostics of a disease. Preferably, the disease is selected from the group consisting of diseases of the central nervous system, preferably neurodegenerative diseases; more preferably Alzheimer's disease, Parkinson's disease, frontotemporal dementia or Amyotrophic Lateral Sclerosis; neuroinflammatory or autoimmune diseases, preferably Multiple Sclerosis, auto-antibody-mediated encephalitis or infectious diseases, neurovascular diseases; preferably stroke, vasculitis; traumatic brain injury, and cancer.

Further, the present invention is directed to the use of such a microglia cell according to the present invention for in vitro culturing with brain organoids.

### BRIEF DESCRIPTION OF THE FIGURES

FIG. 1 shows a scheme of major pathways for cell manufacturing, that are reprogramming of somatic cells (fibroblasts) into induced pluripotent stem cells (iPSC) using the four defined transcription factors Klf4, Oct4, c-Myc and Sox2, direct reprogramming as direct conversion of somatic cells into the desired target cell type using defined transcription factors, classical differentiation approaches, representing a stepwise conversion from a pluripotent stem cell into the desired target cell, and forward programming as the direct conversion of hPSCs into the target cell type. (Abbreviations: TF=transcription factor, ESC=embryonic stem cell, iPSC=induced pluripotent stem cell (ESCs and iPSCs are collectively termed pluripotent stem cells (PSCs))

FIG. 2 shows the targeting strategy used in the present invention. The dox inducible Tet-ON system was targeted into the human ROSA26 locus (CAG-rtTA) and the AAVS1 site (TRE-EGFP) of hPSCs. (Abbreviations: HAR=homology arm, Neo=neomycin-resistance gene, CAG=constitutive CAG promoter, rtTA=reverse tetracycline-controlled transactivator, Puro=puromycin-resistance gene, TRE=inducible Tet-responsive element, EGFP=enhanced green fluorescent protein, SA=splice acceptor, T2A=T2A cleavage site, pA=poly-adenylation site)

FIG. 3 shows a table of the key transcription factors of the microglia lineage, selected as candidate reprogramming factors, the length of their coding sequence and their source.

FIG. 4 shows donor plasmids that were generated by molecular cloning and used for the genetic modification of either the ROSA26 GSH or the AAVS1 GSH. (Abbreviations: HAR=homology arm, Neo=neomycin-resistance gene, CAG=constitutive CAG promoter, rtTA=reverse tetracycline-controlled transactivator, Puro=puromycin-resistance gene, TRE=inducible Tet-responsive element, EGFP=enhanced green fluorescent protein, SA=splice acceptor, T2A=T2A cleavage site, pA=poly-adenylation site)

FIG. 5 shows a scheme of the microglia forward programming protocol (see FIG. 5A). Time-course of cell surface markers expressed on primitive macrophages and microglia assessed by flow cytometry (n=2 biological replicates) (see FIG. 5B and FIG. 5C). Day 20 microglia monoculture: phase contrast live image of a microglia-like cell and ICC for the microglia-signature transmembrane protein TMEM119, for which dedicated labelled flow-antibodies are not available (see FIG. 5D). Day 20 microglia/neuron coculture: ICC for the intracellular calcium-binding protein IBA1 (also known as AIF1) and the neuronal marker βIII-tubulin (TUBB3) (see FIG. 5E). QPCR (SYBR green) of hiPSCs and microglia in monoculture (day 20). All values are relative to the housekeeping gene GAPDH and normalised to hiPSCs. For the transcripts of SPI1 and CEBPB two different primer pairs (see SEQ ID NOs: 80-87; SEQ ID NO: 80: SPI1 total forward primer; SEQ ID NO: 81: SPI1 total reverse primer; SEQ ID NO: 82: SPI1 endo forward primer; SEQ ID NO: 83: SPI1 endo reverse primer; SEQ ID NO: 84: CEBPB total forward primer; SEQ ID NO: 85: CEBPB total reverse primer; SEQ ID NO: 86: CEBPB endo forward primer; SEQ ID NO: 87: CEBPB endo reverse primer) were used, detecting either all transcripts (total), or only transcripts from the respective endogenous gene loci, but not the AAVS1-targeted transgenes (endo). As expected, no difference was detected in the relative expression levels, as transgene expression was turned off (by withdrawal of dox at day 10 of the protocol), thus confirming the transgene-independence of the cellular phenotype (F).

FIG. 6 shows immunocytochemistry of a double targeted iPS cell line induced with doxycycline for 24 hours. The cells were positive for PU.1 and CEBPB but negative for OCT4.

FIG. 7 shows a map of the Donor Plasmid pUC_AAVS1_p-Resp-(PU.1-CEBPB) (SEQ ID NO: 61), for genetic modification of the AAVS1 locus, containing the coding sequence of the transcription factors PU.1 and CEBPB.

FIG. 8 shows a map of the Donor Plasmid pUC_AAVS1_p-Resp-(PU.1-IRF8) (SEQ ID NO: 62), for genetic modification of the AAVS1 locus, containing the coding sequence of the transcription factors PU.1 and IRF8.

FIG. 9 shows a map of the Donor Plasmid pUC_AAVS1_p-Resp-(PU.1-RUNX1) (SEQ ID NO: 63), for genetic modification of the AAVS1 locus, containing the coding sequence of the transcription factors PU.1 and RUNX1.

FIG. 10 shows a map of the Donor Plasmid pUC_AAVS1_p-Resp-(PU.1) (SEQ ID NO: 64), for genetic modification of the AAVS1 locus, containing the coding sequence of the transcription factor PU.1.

FIG. 11 shows a map of the Donor Plasmid pUC_AAVS1_p-Resp-(PU.1-SALL1) (SEQ ID NO: 65), for genetic modification of the AAVS1 locus, containing the coding sequence of the transcription factors PU.1 and SALL1.

FIG. 12 shows a map of the plasmid ROSA-guideA_Cas9n (SEQ ID NO: 66) containing the coding sequence of the Cas enzyme and guide RNA A.

FIG. 13 shows a map of the plasmid ROSA-guideB_Cas9n (SEQ ID NO: 67) containing the coding sequence of the Cas enzyme and guide RNA B.

FIG. 14 shows a map of the donor plasmid pUC_ROSA_n_CAG-rtTA (SEQ ID NO: 72) containing the constitutive CAG promoter and the rtTA.

FIG. 15 shows a map of the plasmid pZFN-AAVS1-L_ELD (SEQ ID NO: 68).

FIG. 16 shows a map of the plasmid pZFN-AAVS1-R_KKR (SEQ ID NO: 69).

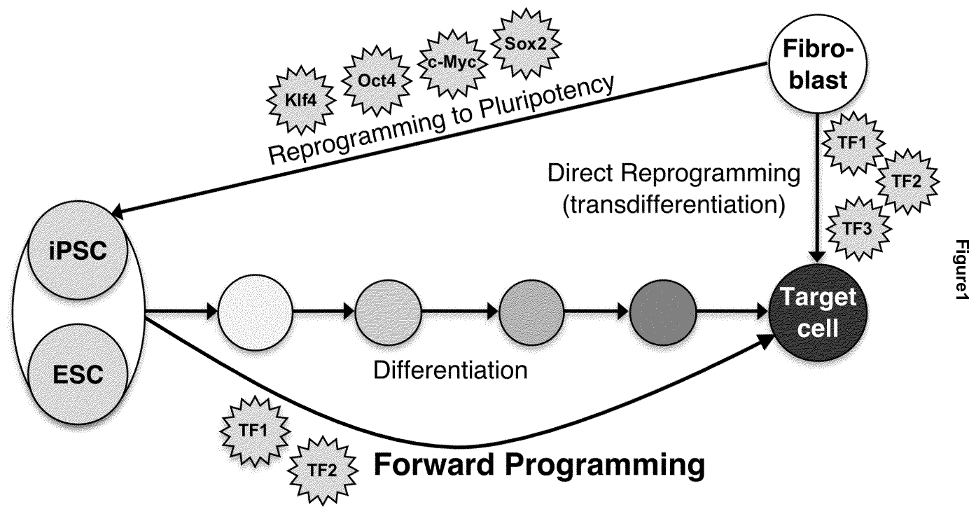

The following abbreviations are used: T2A: T2A peptide (ribosomal skipping signal), puroR: puromycin resistance gene, pA: polyadenylation signal, CAG: constitutive CAG promoter, TRE3GV: Tet-responsive element, HA-R, HA-L: homology arm (right, left), AmpR: Ampicillin resistance gene, ori: origin of replication, NeoR: neomycin resistance gene, KanR: kanamycin resistance gene.

### DETAILED DESCRIPTION OF THE INVENTION

The present invention relates to a method for the production of microglia from stem cells, comprising the steps of a) targeted insertion of a nucleotide sequence encoding a transcriptional regulator protein into a first genomic safe harbour site; and b) targeted insertion of the coding sequence of the transcription factor PU.1 (SEQ ID NO: 1) into a second genomic safe harbour site, wherein the gene is operably linked to an inducible promoter, which is regulated by the transcriptional regulator protein; expression of PU.1 (SEQ ID NO: 2); and c) culturing the stem cells received from steps a) and b) with exposure to at least one growth factor or small molecule that recapitulates signaling during at least one stage of embryonic development of microglia or adult microglia proliferation, differentiation or polarization.

In one embodiment, the present invention relates to a method of producing microglia from stem cells, comprising the steps of a) targeted insertion of a nucleotide sequence encoding a transcriptional regulator protein into a first genomic safe harbour site; and b) targeted insertion of the coding sequence of the transcription factor PU.1 (SEQ ID NO: 1) into a second genomic safe harbour site, wherein the gene is operably linked to an inducible promoter, which is regulated by the transcriptional regulator protein; expression of PU.1 (SEQ ID NO: 2); and c) culturing the stem cells received from steps a) and b) with exposure to at least one growth factor or small molecule that recapitulates signaling during at least one stage of embryonic development of microglia or adult microglia proliferation, differentiation or polarization.

In one embodiment, the present invention relates to a method of producing microglia from stem cells, comprising the steps of a) targeted insertion of a nucleotide sequence encoding a transcriptional regulator protein into a first genomic safe harbour site; and b) targeted insertion of the coding sequence of the transcription factor PU.1 (SEQ ID NO: 1) into a second genomic safe harbour site, wherein the gene is operably linked to an inducible promoter, which is regulated by the transcriptional regulator protein; expression of PU.1 (SEQ ID NO: 2); and c) culturing the stem cells received from steps a) and b) with exposure to at least one growth factor or small molecule that recapitulates signaling during at least one stage of embryonic development of microglia.

In one embodiment, the present invention relates to a method for the production of microglia from stem cells, comprising the steps of a) targeted insertion of a nucleotide sequence encoding a transcriptional regulator protein into a first genomic safe harbour site; and b) targeted insertion of the coding sequence of the transcription factor PU.1 (SEQ ID NO: 1) into a second genomic safe harbour site, wherein the gene is operably linked to an inducible promoter, which is regulated by the transcriptional regulator protein; expression of PU.1 (SEQ ID NO: 2); and c) culturing the stem cells received from steps a) and b) with exposure to at least one growth factor or small molecule that recapitulates signaling during at least one stage of embryonic development of microglia.

In one embodiment, the present invention also relates to a method for the production of microglia from stem cells, comprising the steps of a) targeted insertion of a nucleotide sequence encoding a transcriptional regulator protein into a first genomic safe harbour site; and b) targeted insertion of the coding sequence of the transcription factor PU.1 (SEQ ID NO: 1) into a second genomic safe harbour site, wherein the gene is operably linked to an inducible promoter, which is regulated by the transcriptional regulator protein; expression of PU.1 (SEQ ID NO: 2); and c) culturing the stem cells received from steps a) and b) with exposure to at least one growth factor or small molecule that recapitulates signaling during at least one stage of adult microglia differentiation.

In one further embodiment, the present invention relates to a method for the production of microglia from stem cells, comprising the steps of a) targeted insertion of a nucleotide sequence encoding a transcriptional regulator protein into a first genomic safe harbour site; and b) targeted insertion of the coding sequence of the transcription factor PU.1 (SEQ ID NO: 1) into a second genomic safe harbour site, wherein the gene is operably linked to an inducible promoter, which is regulated by the transcriptional regulator protein; expression of PU.1 (SEQ ID NO: 2); and c) culturing the stem cells received from steps a) and b) with exposure to at least one growth factor or small molecule that recapitulates signaling during at least one stage of adult microglia polarization.

In another embodiment, the present invention relates to a method for the production of microglia from stem cells, comprising the steps of a) targeted insertion of a nucleotide sequence encoding a transcriptional regulator protein into a first genomic safe harbour site; and b) targeted insertion of the coding sequence of the transcription factor PU.1 (SEQ ID NO: 1) into a second genomic safe harbour site, wherein the gene is operably linked to an inducible promoter, which is regulated by the transcriptional regulator protein; expression of PU.1 (SEQ ID NO: 2); and c) culturing the stem cells received from steps a) and b) with exposure to at least one growth factor or small molecule that recapitulates embryonic development of microglia.

In one further embodiment, the present invention relates to a method for the production of microglia from stem cells, comprising the steps of a) targeted insertion of a nucleotide sequence encoding a transcriptional regulator protein into a first genomic safe harbour site; and b) targeted insertion of the coding sequence of the transcription factor PU.1 (SEQ ID NO: 1) into a second genomic safe harbour site, wherein the gene is operably linked to an inducible promoter, which is regulated by the transcriptional regulator protein; expression of PU.1 (SEQ ID NO: 2); and c) culturing the stem cells received from steps a) and b) with exposure to at least one growth factor or small molecule that mimics signaling during at least one stage of embryonic development of microglia or adult microglia proliferation, differentiation or polarization.

In one further embodiment, the present invention relates to a method for the production of microglia from stem cells, comprising the steps of a) targeted insertion of a nucleotide sequence encoding a transcriptional regulator protein into a first genomic safe harbour site; and b) targeted insertion of the coding sequence of the transcription factor PU.1 (SEQ ID NO: 1) into a second genomic safe harbour site, wherein the gene is operably linked to an inducible promoter, which is regulated by the transcriptional regulator protein; expression of PU.1 (SEQ ID NO: 2); and c) culturing the stem cells received from steps a) and b) with exposure to at least one growth factor or small molecule that mimics signaling during at least one stage of embryonic development of microglia.

In one embodiment, the present invention also relates to a method for the production of microglia from stem cells, comprising the steps of a) targeted insertion of a nucleotide sequence encoding a transcriptional regulator protein into a first genomic safe harbour site; and b) targeted insertion of the coding sequence of the transcription factor PU.1 (SEQ ID NO: 1) into a second genomic safe harbour site, wherein the gene is operably linked to an inducible promoter, which is regulated by the transcriptional regulator protein; expression of PU.1 (SEQ ID NO: 2); and c) culturing the stem cells received from steps a) and b) with exposure to at least one growth factor or small molecule that recapitulates embryonic development of microglia in vitro.

As used within the present invention, the term “microglia” means a mature cell type being a distinct cell population of the central nervous system. As defined in Comparative Anatomy and Histology, “microglia is the resident histiocytic-type cell and the key innate immune effector of the CNS. They are often described as either resting (i.e., ramified) or activated, but these terms fail to convey the dynamic remodeling of their fine processes and constitutive immunosurveillance activity. ( . . . ) Evidence suggests that early microglia are derived from yolk sac progenitors.” (Hagan et al., 2012). Meaning microglia are generated during early embryonic stages and reside in the brain throughout adult live.

As used within the present invention, the term “production of microglia” means the generation of a mature cell (microglia) from a stem cell, which is obtained by any of the methods of the present invention as described herein.

As used within the present invention, the term “stem cell” means a type of cell that is able to divide for producing more cells or to develop into a cell that has a particular purpose. In the present invention, the used stem cell might be a pluripotent stem cell. Pluripotent stem cells have the potential to differentiate into almost any cell in the body. There are several sources of pluripotent stem cells. Embryonic stem cells (ES cells) are pluripotent stem cells derived from the inner cell mass of a blastocyst, an early-stage preimplantation embryo. Induced pluripotent stem cells (iPSCs) are adult cells that have been genetically reprogrammed to an embryonic stem cell-like state by being forced to express genes and factors important for maintaining the defining properties of embryonic stem cells. In 2006 it was shown that the introduction of four specific genes encoding transcription factors could convert adult cells into pluripotent stem cells (Takahashi et al., 2006), but subsequent work has reduced/altered the number of genes that are required. Oct-3/4 and certain members of the Sox gene family have been identified as potentially crucial transcriptional regulators involved in the induction process. Additional genes including certain members of the Klf family, the Myc family, Nanog, and LIN28, may increase the induction efficiency. Examples of the genes, which may be contained in the reprogramming factors, include Oct3/4, Sox2, SoxI, Sox3, SoxI5, SoxI7, Klf4, Klf2, c-Myc, N-Myc, L-Myc, Nanog, Lin28, Fbxl5, ERas, ECAT15-2, Tell, beta-catenin, Lin28b, Sall1, Sall4, Esrrb, Nr5a2, Tbx3 and GlisI, and these reprogramming factors may be used singly, or in combination of two or more kinds thereof.

If the cells modified by insertion of an inducible cassette are to be used in a human patient, it may be preferred that the cell is an iPSC derived from that individual. Such use of autologous cells would remove the need for matching cells to a recipient. Alternatively, commercially available iPSC may be used, which are known to a person skilled in the art. Alternatively, the cells may be a tissue-specific stem cell, which may also be autologous or donated. Suitable cells include epiblast stem cells, induced neural stem cells and other tissue-specific stem cells.

In some embodiments of the method of the present invention, it may be preferred that the used stem cell is an embryonic stem cell or stem cell line. Numerous embryonic stem cell lines are now available, for example, WA01 (HI), WA09 (H9), KhES-1, KhES-2 and KhES-3. Stem cell lines, which have been derived without destroying an embryo, are available. The present invention does not extend to any methods which involve the destruction of human embryos.

As used within the present invention, the term “targeted insertion” means the insertion into a genomic safe harbour (GSH) site, which is preferably specifically within the sequence of the GSH as described elsewhere. Any suitable technique for insertion of a polynucleotide into a specific sequence may be used, and several are described in the art. Suitable techniques include any method known to a person skilled in the art, which introduces a break at the desired location and permits recombination of the vector into the gap. Thus, a crucial first step for targeted site-specific genomic modification is the creation of a double-strand DNA break (DSB) at the genomic locus to be modified. Distinct cellular repair mechanisms can be exploited to repair the DSB and to introduce the desired sequence, and these are non-homologous end joining repair (NHEJ), which is more prone to error; and homologous recombination repair (HR) mediated by a donor DNA template, that can be used to insert inducible cassettes.

Several techniques exist to allow customized site-specific generation of DSB in the genome. Many of these involve the use of customized endonucleases, such as zinc finger nucleases (ZFNs), transcription activator-like effector nucleases (TALENs) or the clustered regularly interspaced short palindromic repeats/CRISPR associated protein (CRISPR/Cas9) system (Gaj et al., 2013). Zinc finger nucleases are artificial enzymes, which are generated by fusion of a zinc-finger DNA-binding domain to the nuclease domain of the restriction enzyme FokI. The latter has a non-specific cleavage domain, which must dimerize in order to cleave DNA. This means that two ZFN monomers are required to allow dimerization of the FokI domains and to cleave the DNA. The DNA binding domain may be designed to target any genomic sequence of interest, may be a tandem array of Cys2His2 zinc fingers, each of which recognises three contiguous nucleotides in the target sequence. The two binding sites are separated by 5-7 bp to allow optimal dimerization of the FokI domains. The enzyme thus is able to cleave DNA at a specific site, and target specificity is increased by ensuring that two proximal DNA-binding events must occur to achieve a double-strand break. Transcription activator-like effector nucleases, or TALENs, are dimeric transcription factor/nucleases. They are made by fusing a TAL effector DNA-binding domain to a DNA cleavage domain (a nuclease). Transcription activator-like effectors (TALENs) can be engineered to bind practically any desired DNA sequence, so when combined with a nuclease, DNA can be cut at specific locations. TAL effectors are proteins that are secreted by Xanthomonas bacteria, the DNA binding domain of which contains a repeated highly conserved 33-34 amino acid sequence with divergent 12th and 13th amino acids. These two positions are highly variable and show a strong correlation with specific nucleotide recognition. This straightforward relationship between amino acid sequence and DNA recognition has allowed for the engineering of specific DNA-binding domains by selecting a combination of repeat segments containing appropriate residues at the two variable positions. TALENs are thus built from arrays of 33 to 35 amino acid modules, each of which targets a single nucleotide. By selecting the array of the modules, almost any sequence may be targeted. The nuclease used may be FokI or a derivative thereof.

Three types of CRISPR mechanisms have been identified, of which type II is best studied. The CRISPR/Cas9 system (type II system) utilises the Cas9 nuclease to make a double-stranded break in DNA at a site determined by a short guide RNA. The CRISPR/Cas system is a prokaryotic immune system that confers resistance to foreign genetic elements. CRISPR are segments of prokaryotic DNA containing short repetitions of base sequences. Each repetition is followed by short segments of “protospacer DNA” from previous exposures to foreign genetic elements. CRISPR spacers recognize and cut the exogenous genetic elements using RNA interference. The CRISPR immune response occurs through two steps: CRISPR-RNA (crRNA) biogenesis and crRNA-guided interference. CrRNA molecules are composed of a variable sequence transcribed from the protospacer DNA and a CRISP repeat. Each crRNA molecule then hybridizes with a second RNA, known as the trans-activating CRISPR RNA (tracrRNA) and together these two eventually form a complex with the nuclease Cas9. The protospacer DNA encoded section of the crRNA directs Cas9 to cleave complementary target DNA sequences, if they are adjacent to short sequences known as protospacer adjacent motifs (PAMs). This natural system has been engineered and exploited to introduce DSB breaks in specific sites in genomic DNA, amongst many other applications. In particular, the CRISPR type II system from Streptococcus pyogenes may be used. At its simplest, the CRISPR/Cas9 system comprises two components that are delivered to the cell to provide genome editing: The Cas9 nuclease itself and a small guide RNA (gRNA). The gRNA is a fusion of a customised, site-specific crRNA (directed to the target sequence) and a standardized tracrRNA.

Once a DSB has been made, a donor template with homology to the targeted locus is supplied. The DSB may be repaired by the homology-directed repair (HDR) pathway allowing for precise insertions to be made. Derivatives of this system are also possible. Mutant forms of Cas9 are available, such as Cas9D10A, with only nickase activity. This means, it cleaves only one DNA strand, and does not activate NHEJ. Instead, when provided with a homologous repair template, DNA repairs are conducted via the high-fidelity HDR pathway only. Cas9D10A may be used in paired Cas9 complexes designed to generate adjacent DNA nicks in conjunction with two sgRNAs, complementary to the adjacent area on opposite strands of the target site, which may be particularly advantageous. The elements for making the double-strand DNA break may be introduced in one or more vectors such as plasmids for expression in the cell. Thus, any method of making specific, targeted double strand breaks in the genome in order to allow the insertion of a nucleotide sequence/gene/inducible cassette may be used in the method of the present invention. It may be preferred that the method of the present invention utilises for inserting the gene/inducible cassette any one or more of ZFNs, TALENs and/or CRISPR/Cas9 systems or any derivative thereof.

Once the DSB has been made by any appropriate means, the gene/inducible cassette for insertion may be supplied in any suitable fashion as described below. The gene/inducible cassette and associated genetic material form the donor DNA for repair of the DNA at the DSB and are inserted using standard cellular repair machinery/pathways. How the break is initiated will alter which pathway is used to repair the damage, as noted above. However, this is also within the knowledge of a person skilled in the art.

As used within the present invention, the term “gene” means the basic physical unit heredity, a linear sequence of nucleotides along a segment of DNA that provides the coded instructions for synthesis of RNA, which, when translated into protein, leads to the expression of hereditary character.

As used within the present invention, the term “nucleotide sequence” refers to a succession of bases in a DNA segment forming a gene as defined above.

As used within the present invention, the term “transcriptional regulator protein” means a protein that binds to DNA, preferably sequence-specifically to a DNA site located in or near a promoter, and either facilitating the binding of the transcription machinery to the promoter, and thus transcription of the DNA sequence (a transcriptional activator) or blocks this process (a transcriptional repressor). Such entities are also known as transcription factors. The DNA sequence that a transcriptional regulator protein binds to is called a transcription factor-binding site or response element, and these are found in or near the promoter of the regulated DNA sequence. A responsive element is part of this invention. Transcriptional activator proteins bind to a response element and promote gene expression. Such proteins are preferred in the method of the present invention for controlling inducible cassette expression. Transcriptional repressor proteins bind to a response element and prevent gene expression. Transcriptional regulator proteins may be activated or deactivated by a number of mechanisms including binding of a substance, interaction with other transcription factors (e.g., homo- or hetero-dimerization) or coregulatory proteins, phosphorylation, and/or methylation. The transcriptional regulator may be controlled by activation or deactivation. If the transcriptional regulator protein is a transcriptional activator protein, it is preferred that the transcriptional activator protein requires activation. This activation may be through any suitable means, but it is preferred that the transcriptional regulator protein is activated through the addition of an exogenous substance to the stem cell. The supply of an exogenous substance to the stem cell can be controlled, and thus the activation of the transcriptional regulator protein can be controlled. Such transcriptional regulator proteins are also called inducible transcriptional regulator proteins.

As used within the present invention, the term “transcription factor” means a protein that binds to DNA, preferably sequence-specifically to a DNA site located in or near a promoter, and either facilitating the binding of the transcription machinery to the promoter, and thus transcription of the DNA sequence (a transcriptional activator) or blocks this process (a transcriptional repressor). In the context of the present invention, a transcription factor is a desired genetic sequence, preferably a DNA sequence that is to be transferred into a cell together with an inducible cassette. The introduction of an inducible cassette into the genome has the potential to change the phenotype of that cell by addition of a genetic sequence that permits gene expression. The method of the present invention provides for controllable transcription of the genetic sequence(s) of a set of transcription factors within the inducible cassette in the cell.

Master regulators may be one or more of: transcription factors, transcriptional regulators, cytokine receptors or signalling molecules and the like. A master regulator is an expressed gene that influences the lineage of the cell expressing it. It may be that a network of master regulators is required for the lineage of a cell to be determined. As used herein, a master regulator gene that is expressed at the inception of a developmental lineage or cell type, participates in the specification of that lineage by regulating multiple downstream genes either directly or through a cascade of gene expression changes. If the master regulator is expressed it has the ability to re-specify the fate of cells destined to form other lineages. The transcription factors, which may be used in the method of the present invention, include PU.1 (SEQ ID NO: 2) (gene SPI1, SEQ ID NO: 1), CEBPB (SEQ ID NO: 3), RUNX1 (SEQ ID NO: 4), IRF8 (SEQ ID NO: 5), and SALL1 (SEQ ID NO: 6).

As used within the present invention, the term “PU.1” (SEQ ID NO: 2) means a transcription factor also known as Hematopoietic Transcription Factor PU.1, Spi-1 Proto-Oncogene, 31 kDa Transforming Protein, Transcription Factor PU.1, Spleen Focus Forming Virus (SFFV) Proviral Integration Oncogene Spi1, Spleen Focus Forming Virus (SFFV) Proviral Integration Oncogene, or 31 kDa-Transforming Protein, SFPI1, SPI-1, SPI-A, PU.1 or OF, wherein “SPI1” refers to the gene (SEQ ID NO: 1) (Spi-1 Proto-Oncogene), which encodes an ETS-domain transcription factor that activates gene expression during myeloid and B-lymphoid cell development.

As used within the present invention, the term “genomic safe harbour site” means a genetic site, which allows the insertion of genetic material without deleterious effects for the cell and permits transcription of the inserted genetic material. Those skilled in the art may use these simplified criteria to identify a suitable GSH, and/or the more formal criteria. Insertions specifically within genomic safe harbour sites (GSH) are preferred over random genome integration, since this is expected to be a safer modification of the genome, and is less likely to lead to unwanted side effects, such as silencing natural gene expression or causing mutations that lead to cancerous cell types. Thus, a genomic safe harbour site is a locus within the genome, wherein a gene or other genetic material may be inserted without any deleterious effects on the cell or on the inserted genetic material. Most beneficial is a GSH site in which expression of the inserted gene sequence is not perturbed by any read-through expression from neighbouring genes and expression of the inducible cassette, minimizes interference with the endogenous transcription programme. More formal criteria have been proposed that assist in the determination of whether a particular locus is a GSH site (Pellenz et al., 2019). These criteria include a site that is (i) >300 kb from any cancer-related gene on all Oncogenes list, (ii) >300 kb from any miRNA/other functional small RNAs, (iii) >50 kb from any 5′ gene end, (iv) >50 kb away from any replication origin, (v) >50 kb away from any ultra-conserved element, (vi) low transcriptional activity (no mRNA±25 kb), (vii) not in copy number variable region (viii) in open chromatin (DHS signal±1 kb) and (ix) unique (1 copy in human genome). It may not be necessary to satisfy all of these proposed criteria, since GSH already identified do not fulfil all of these criteria. It is preferred, that a suitable GSH may satisfy at least 3, 4, 5, 6, 7 or 8 and most preferably all nine of these criteria.

In the methods of the present invention, insertions occur at different GSH. At least two GSH are required. The first GSH is modified by insertion of a transcriptional regulator protein. The second GSH is modified by the insertion of an inducible cassette, which comprises a coding sequence operably linked to an inducible promoter. Other genetic material may also be inserted with either or both of these elements. The genetic sequence, operably linked to an inducible promoter within the inducible cassette, is preferably a DNA sequence. The genetic sequence(s) of the inducible cassette preferably encode a RNA molecule and are thus capable of being transcribed. The transcription is controlled using the inducible promoter. The RNA molecule may be of any sequence, but is preferably an mRNA encoding a protein, a shRNA or a gRNA.

The first GSH can be any suitable GSH site. Optionally, it is a GSH with an endogenous promoter that is constitutively expressed, which will result in the inserted transcriptional regulator protein being constitutively expressed. A suitable GSH is the hROSA26 site for human cells. In a further embodiment of the present invention, the inserted transcriptional regulator protein, operably linked to a promoter, is a constitutive promoter. A constitutive promoter can be, for example, used in conjunction with an insertion in the hROSA26 site.

As used within the present invention, the term “inducible promoter” means a nucleotide sequence, which initiates and regulates transcription of a polynucleotide. An “inducible promoter” is a nucleotide sequence, wherein expression of a genetic sequence operably linked to the promoter is controlled by an analyte, co-factor, regulatory protein, etc. In one embodiment of the method of the present invention, the control is affected by the transcriptional regulator protein. It is intended that the term “promoter” or “control element” includes full-length promoter regions and functional (e.g., controls transcription or translation) segments of these regions. It is preferred that the gene encoding the transcriptional regulator protein is operably linked to a constitutive promoter. Alternatively, the first GSH can be selected such that it already has a constitutive promoter that can also drive expression of the transcriptional regulator protein gene and any associated genetic material. Constitutive promoters ensure sustained and high-level gene expression. Commonly used constitutive promoters include the human β-actin promoter (ACTB), cytomegalovirus (CMV), elongation factor-Ia (EFIa), phosphoglycerate kinase (PGK) and ubiquitin C (UbC). The CAG promoter is a strong synthetic promoter frequently used to drive high levels of gene expression.

As used within the present invention, the term “culturing” means the growth of microorganisms such as bacteria and yeast, or human, plant, or animal cells under suitable conditions ensuring the growth, which are knowledge of the person skilled in the art.

As used within the present invention, the term “growth factor” means a signaling molecule that controls cell activities in an autocrine, paracrine or endocrine manner. As used herein, in the context of the present invention, the term “growth factor” may be used interchangeably with “cytokine”. Growth factors or cytokines are produced by different cell types of the organism and exert their biological functions by binding to specific receptors and activating associated downstream signaling pathways which in turn, regulate gene transcription in the nucleus and ultimately stimulate a biological response, including regulatory cellular processes like cell division, cell survival, cell differentiation, adhesion and migration.

As used within the present invention, the term “small molecule” means a bioactive molecule that is naturally or artificially produced and is capable of diffusion through the cell membrane and is able to regulate signaling pathways. Small molecules, which are preferably used within the present invention, may inhibit phosphatidylinositol 3-kinase (PI3K) and glycogen synthase kinase 3, respectively like LY294002 and CHIR99021.

As used within the present invention, the term “recapitulates signaling” means to simulate, to imitate or to resemble the functions of secreted molecules, such as growth factors and/or chemokines, influencing a cell in a natural environment and thereby being able to produce microglia by these actions.

As used within the present invention, the term “mimics signaling” means to simulate, to imitate, to resemble or to recapitulate the functions of secreted molecules, such as growth factors and/or chemokines, influencing a cell in a natural environment and thereby being able to produce microglia by these actions.

As used within the present invention, the term “embryonic development of microglia” means the stepwise transition of a pluripotent stem cell into a mature microglia cell according to the sequel of developmental microglia differentiation during human embryonic, fetal and postnatal development, starting from the pre-implantation blastocyst-stage embryo through to fully-established and self-maintained microglia population.

As used within the present invention, the term “adult microglia proliferation” means any cell division process that leads to a mature microglia cell.

As used within the present invention, the term “adult microglia differentiation” means the differentiation of a cell being in a microglia progenitor's state into an adult microglia cell type, that incorporates typical characteristics of a microglia cell in homeostatic/resting state.

As used within the present invention, the term “adult microglia polarization” means the reaction of a mature microglia cell to extracellular stimuli provided by the extracellular environment, respectively signals from injured neurons, glia cells, or exposure to plasma proteins, due to blood brain barrier dysfunction. This microglial reaction includes movement of the microglia cell towards the injury site and can either have a neuroprotective or -toxic effect.

Further, in one embodiment of the method of the present invention, the at least one growth factor or small molecule is selected from the group consisting of Activin A (SEQ ID NO: 7), BMP4 (SEQ ID NO: 8), FGF (SEQ ID NO: 9), VEGF-A (SEQ ID NO: 10), LY294002, CHIR99021, SCF (SEQ ID NO: 11), IL-3 (SEQ ID NO: 12), IL-6 (SEQ ID NO: 13), CSF1 (SEQ ID NO: 14), IL-34 (SEQ ID NO: 15), CSF2 (SEQ ID NO: 16), CD200 (SEQ ID NO: 17), CX3CL1 (SEQ ID NO: 18), TGFβ1 (SEQ ID NO: 19), and IDE1.

Activin A (SEQ ID NO: 7), as used in the present invention, means Activin beta-A chain, EDF, Erythroid differentiation protein, FRP, FSH-releasing protein, INHBA, Inhibin beta-A chain, Inhibin beta-1. The protein encoded by this gene is a member of the transforming growth factor beta (TGF-β) family of proteins produced by pluripotent stem cells, endoderm, and mesoderm.

BMP4 (SEQ ID NO: 8), as used in the present invention, means bone morphogenetic protein 4, also known as ZYME, BMP2B or BMP2B1. The protein encoded by this gene is a member of the bone morphogenetic protein family, which is part of the transforming growth factor-beta superfamily.

FGF (SEQ ID NO: 9), as used in the present invention means fibroblast growth factor. The protein encoded by this gene is a member of a family of cell signaling proteins as described in e.g. Hui et al., 2018.

VEGF-A (SEQ ID NO: 10), as used in the present invention, means vascular endothelial growth factor A also known as VPF, VEGF or MVCD1. The protein encoded by this gene is a member of the PDGF/VEGF growth factor family and a heparin-binding protein. This growth factor induces proliferation and migration of vascular endothelial cells, and is essential for both physiological and pathological angiogenesis.

LY294002, as used in the present invention, means a potent, cell permeable inhibitor of phosphatidylinositol 3-kinase (PI3K) that acts on the ATP binding site of the enzyme (Vlahos et al., 1994). The chemical structure thereof is given in the following:

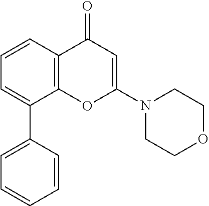 [SMILES: O=c1cc(N2CCOCC2)oc2c(-c3ccccc3)cccc12]

CHIR99021, as used in the present invention, means an amino pyrimidine derivative that is an extremely potent inhibitor of glycogen synthase kinase 3, inhibiting GSK3β (IC₅₀=6.7 nM) and GSK3α (IC₅₀=10 nM) and functions as a WNT activator. The chemical structure thereof is given in the following:

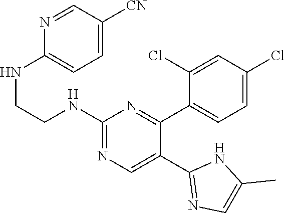 [SMILES: Cc1cnc(-c2cnc(NCCNc3ccc(C#N)cn3)nc2-c2ccc(Cl)cc2Cl)[nH]1]

SCF (SEQ ID NO: 11), as used in the present invention, means Stem cell factor also known as Kit ligand, Mast cell growth factor or Steel factor. The protein encoded by this gene is an early-acting cytokine that plays a pivotal role in the regulation of embryonic and adult hematopoiesis.

IL-3 (SEQ ID NO: 12), as used in the present invention, means Interleukin-3, MCGF (Mast cell growth factor), Multi-CSF, HCGF, P-cell stimulation factor, MGC79398 or MGC79399. The protein encoded by this gene is a growth promoting cytokine.

IL-6 (SEQ ID NO: 13), as used in the present invention, means Interleukin 6 also known as B-Cell Stimulatory Factor 2, CTL Differentiation Factor, Hybridoma Growth Factor, Interferon Beta-2, Interleukin-6, IFN-βeta-2, IFNB2, BSF-2, CDF, Interferon, Beta 2, B-Cell Differentiation Factor, Interferon, Beta 2, Interleukin BSF-2, BSF2, HGF, or HSF. The protein encoded by this gene is a cytokine that functions in inflammation and the maturation of B cells.

CSF1 (SEQ ID NO: 14), as used in the present invention, means Colony Stimulating Factor 1 also known as Colony Stimulating Factor 1 (Macrophage), Macrophage Colony-Stimulating Factor 1, Macrophage Colony Stimulating Factor 1, Lanimostim, CSF-1, MCSF, M-CSF and the protein encoded by this gene is a cytokine that controls the production, differentiation, and function of macrophages.

IL-34 (SEQ ID NO: 15), as used in the present invention, means Interleukin 34, also known as C16 or f77. The protein encoded by this gene is a cytokine that promotes the differentiation and viability of monocytes and macrophages through the colony-stimulating factor-1 receptor.

CSF2 (SEQ ID NO: 16), as used in the present invention, means Colony Stimulating Factor 2 also known as Sargramostim, Colony Stimulating Factor 2 (Granulocyte-Macrophage), Granulocyte-Macrophage Colony-Stimulating Factor, Molgramostin, Molgramostim, GMCSF, CSF, Granulocyte Macrophage-Colony Stimulating Factor, Granulocyte-Macrophage Colony Stimulating Factor, Colony-Stimulating Factor, GM-CSF. The protein encoded by this gene is a cytokine that controls the production, differentiation, and function of granulocytes and macrophages.

CD200 (SEQ ID NO: 17), as used in the present invention, means the CD200 Gene also known as CD200 Molecule, CD200 Antigen, Antigen Identified by Monoclonal Antibody MRC OX-2, OX-2 Membrane Glycoprotein, MOX1, MOX2, OX-2 or MRC. The protein encoded by this gene is a type I membrane glycoprotein containing two extracellular immunoglobulin domains, a transmembrane and a cytoplasmic domain.

CX3CL1 (SEQ ID NO: 18), as used in the present invention, means the CX3CL1 Gene also known as C-X3-C Motif Chemokine Ligand 1, Small Inducible Cytokine Subfamily D (Cys-X3-Cys), Member 1 (Fractalkine, Neurotactin), Chemokine (C-X3-C Motif) Ligand 1, CX3C Membrane-Anchored Chemokine, Small-Inducible Cytokine D1, C-X3-C Motif Chemokine 1, Neurotactin, Fractalkine, or SCYD1, NTT, Small Inducible Cytokine Subfamily D (Cys-X3-Cys), Member-1, C3Xkine, ABCD-3, CXC3C, CXC3, NTN or FKN. The protein encoded by this gene belongs to the CX3C subgroup of chemokines, characterized by the number of amino acids located between the conserved cysteine residues.

TGFβ1 (SEQ ID NO: 19), as used in the present invention, means the Transforming Growth Factor Beta 1, also known as Transforming Growth Factor Beta-1 Proprotein, Prepro-Transforming Growth Factor Beta-1, TGFB, Transforming Growth Factor, Beta 1, Transforming Growth Factor Beta-1, Latency-Associated Peptide, Camurati-Engelmann Disease, TGF-Beta-1, IBDIMDE, TGFbeta, DPD1, CED or LAP. The protein encoded by this gene is a secreted ligand of the TGF-beta (transforming growth factor-beta) superfamily of proteins.

In a further embodiment of the method of the present invention, the at least one growth factor is CSF1 (SEQ ID NO: 14) or IL-34 (SEQ ID NO: 15). In a further embodiment of the method of the present invention, the at least one growth factor is CSF1 (SEQ ID NO: 14). In a further embodiment of the method of the present invention, the at least one growth factor is IL-34 (SEQ ID NO: 15).

In an additional embodiment of the method of the present invention, the at least one small molecule is CHIR99021, LY294002 or IDE1.

LY294002, as used in the present invention, means a potent, cell permeable inhibitor of phosphatidylinositol 3-kinase (PI3K) that acts on the ATP binding site of the enzyme (Vlahos et al., 1994). The chemical structure thereof is given in the following:

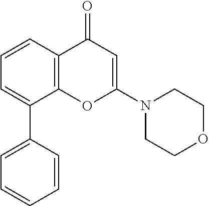 [SMILES: O=c1cc(N2CCOCC2)oc2c(-c3ccccc3)cccc12]

CHIR99021, as used in the present invention, means an amino pyrimidine derivative that is a potent inhibitor of glycogen synthase kinase 3, inhibiting GSK3β (IC₅₀=6.7 nM) and GSK3α (IC₅₀=10 nM) and functions as a WNT activator. The chemical structure thereof is given in the following:

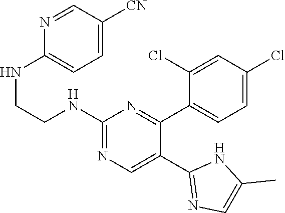 [SMILES: Cc1cnc(-c2cnc(NCCNc3ccc(C#N)cn3)nc2-c2ccc(Cl)cc2Cl)[nH]1]

IDE1, as used in the present invention, means inducer of definitive endoderm; a small molecule that activates the TGF-beta pathway and could be used as a replacement of the growth factor TGF-beta. The chemical structure thereof is given in the following:

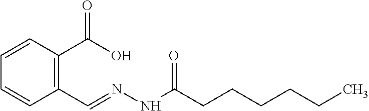 [SMILES: CCCCCCC(=O)N/N=C/c1ccccc1C(=O)O]

In another embodiment of the method of the present invention, the first and the second genomic safe harbour sites are different.

In a further embodiment of the method of the present invention, the method further comprises insertion of the coding sequence of the gene of the transcription factor CEBPB (SEQ ID NO: 3) and expression thereof.

CEBPB (SEQ ID NO: 3) as used in the present invention means CCAAT Enhancer Binding Protein Beta also known as CCAAT Enhancer Binding Protein Beta, CCAAT/Enhancer Binding Protein (C/EBP), Beta, Interleukin 6-Dependent DNA-Binding Protein, CCAAT/Enhancer-Binding Protein Beta, Nuclear Factor of Interleukin 6, Transcription Factor 5, Nuclear Factor NF-IL6, TCF5, Liver-Enriched Transcriptional Activator Protein, CCAAT/Enhancer Binding Protein Beta, Liver-Enriched Inhibitory Protein, Transcription Factor C/EBP Beta, Liver Activator Protein, C/EBP-Beta, C/EBP Beta, IL6DBP, NF-IL6, TCF-5, LAP or LIP. This intronless gene encodes a transcription factor that contains a basic leucine zipper (bZIP) domain.

In another embodiment of the method of the present invention, the method further comprises insertion of the coding sequence of the gene of the transcription factor RUNX1 (SEQ ID NO: 4) and expression thereof.

RUNX1 (SEQ ID NO: 4) as used in the present invention means Runt Related Transcription Factor 1, Runt-Related Transcription Factor 1, Polyomavirus Enhancer-Binding Protein 2 Alpha B Subunit, SL3/AKV Core-Binding Factor Alpha B Subunit, SL3-3 Enhancer Factor 1 Alpha B Subunit, Acute Myeloid Leukemia 1 Protein, Oncogene AML-1, PEBP2-Alpha B, PEA2-Alpha B, CBFA2, AML1, Core-Binding Factor Runt Domain Alpha Subunit 2 Core-Binding Factor Subunit Alpha-2, AML1-EVI-1 Fusion Protein, Acute Myeloid Leukemia, Aml1 Oncogene, CBF-Alpha-2, AML1-EVI-1, PEBP2alpha, CBF2alpha, PEBP2aB, AMLCR1 or EVI-1. The protein encoded by this gene represents the alpha subunit of CBF and is thought to be involved in the development of normal hematopoiesis.

In a further embodiment of the method of the present invention, the method further comprises insertion of the coding sequence of the gene of the transcription factor IRF8 (SEQ ID NO: 5) and expression thereof.

IRF8 (SEQ ID NO: 5) as used in the present invention means Interferon Regulatory Factor 8, also known as Interferon Consensus Sequence Binding Protein 1, H-ICSBP, ICSBP1, ICSBP, IRF-8, Interferon Consensus Sequence-Binding Protein, IMD32A, IMD32B or Interferon consensus sequence-binding protein (ICSBP). It is a transcription factor of the interferon (IFN) regulatory factor (IRF) family.

In another embodiment of the method of the present invention, the method further comprises insertion of the coding sequence of the gene of the transcription factor SALL1 (SEQ ID NO: 6) and expression thereof.

SALL1 (SEQ ID NO: 6), as used in the present invention, means Spalt Like Transcription Factor 1, also known as Zinc Finger Protein Spalt-1, Zinc Finger Protein SALL1, Zinc Finger Protein 794, Sal-Like Protein 1, ZNF794, Sal-1, Epididymis Secretory Protein Li 89, Spalt-Like Transcription Factor 1, Sal (Drosophila)-Like 1, Sal-Like 1 (Drosophila), HEL-S-89, HSAL1, HSal1, SAL1 or TBS. The protein encoded by this gene is a zinc finger transcriptional repressor and may be part of the NuRD histone deacetylase complex (HDAC).

In an additional embodiment of the method of the present invention, the transcriptional regulator protein is the reverse tetracycline transactivator (rtTA) (SEQ ID NO: 20) and the activity thereof is controlled by doxycycline or tetracycline.

As used within the present invention, the term “reverse tetracycline transactivator (rtTA)” means a transcriptional activator protein induced by tetracycline or a derivate thereof. Tetracycline-controlled transcriptional activation is a method of inducible gene expression where transcription is reversibly turned on or off in the presence of the antibiotic tetracycline or one of its derivatives (e.g. doxycycline, which is more stable). In this system, the transcriptional activator protein may be tetracycline-responsive transcriptional activator protein (rtTa) or a derivative thereof. The transcriptional regulator protein of the present invention may be an rtTA. The rtTA protein is able to bind to DNA at specific TetO operator sequences. Several repeats of such TetO sequences are placed upstream of a minimal promoter (such as the CMV promoter), which together form a tetracycline response element (TRE) (SEQ ID NO: 21). There are two forms of this system, depending on whether the addition of tetracycline or a derivative activates (Tet-On) or deactivates (Tet-Off) the rtTA protein. The Tet-ON system, in which doxycycline activates the rtTA protein, may also be used in one embodiment of the method of the present invention.

The Tet-On system is composed of two components; (1) the constitutively expressed tetracycline-responsive transcriptional activator protein (rtTA) and the rtTA sensitive inducible promoter (Tet Responsive Element, TRE). This may be bound by tetracycline or its more stable derivatives, including doxycycline (dox), resulting in activation of rtTA, allowing it to bind to TRE sequences and inducing expression of TRE-controlled genes. The use of this may be preferred in the method of the present invention. Thus, the transcriptional regulator protein of the method of the present invention may be the tetracycline-responsive transcriptional activator protein (rtTA), which can be activated or deactivated by the antibiotic tetracycline or one of its derivatives, which are supplied exogenously. If the transcriptional regulator protein is rtTA, then the inducible promoter inserted into the second GSH site includes the tetracycline response element (TRE). The exogenously supplied substance may be the antibiotic tetracycline or one of its derivatives, like doxycycline, preferably tetracycline or doxycycline.

Variants and modified rtTA proteins may be used in the method of the present invention. These may include Tet-On Advanced transactivator (also known as rtTA2S-M2) and Tet-On 3G (also known as rtTA-V16, derived from rtTA2S-S2).

In another embodiment of the method of the present invention, the inducible promoter includes a Tet Responsive Element (TRE) (SEQ ID NO: 21).

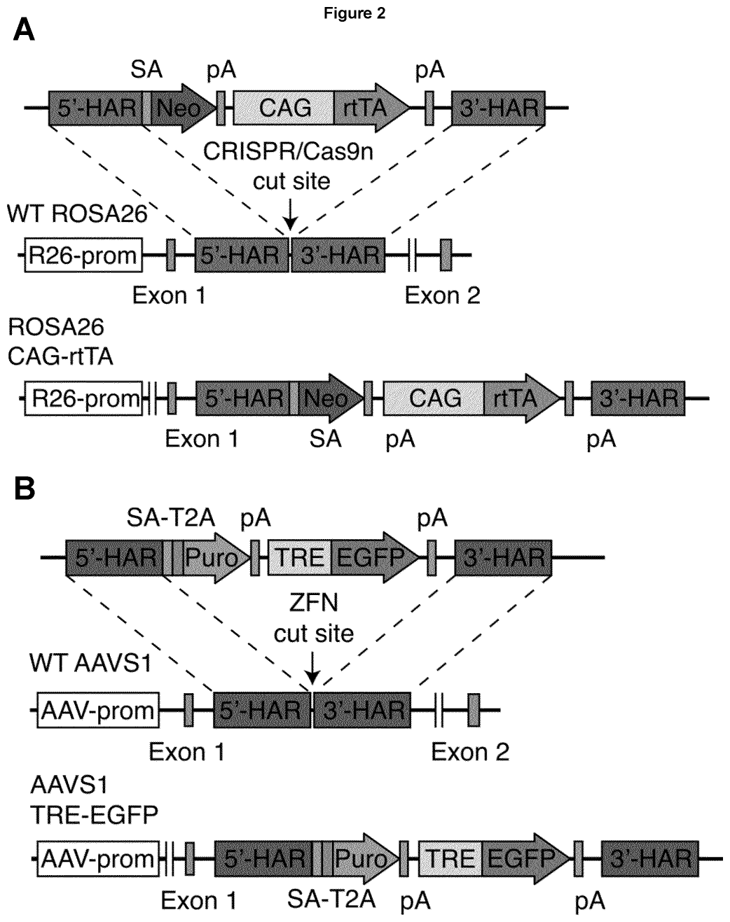

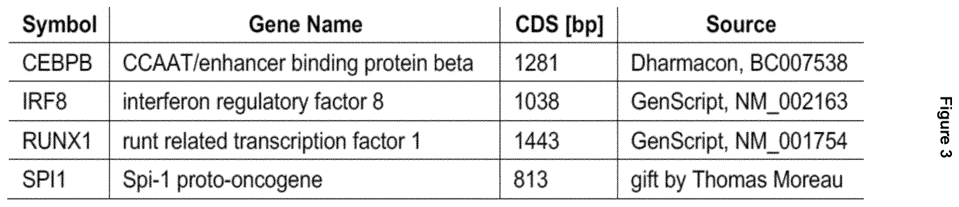

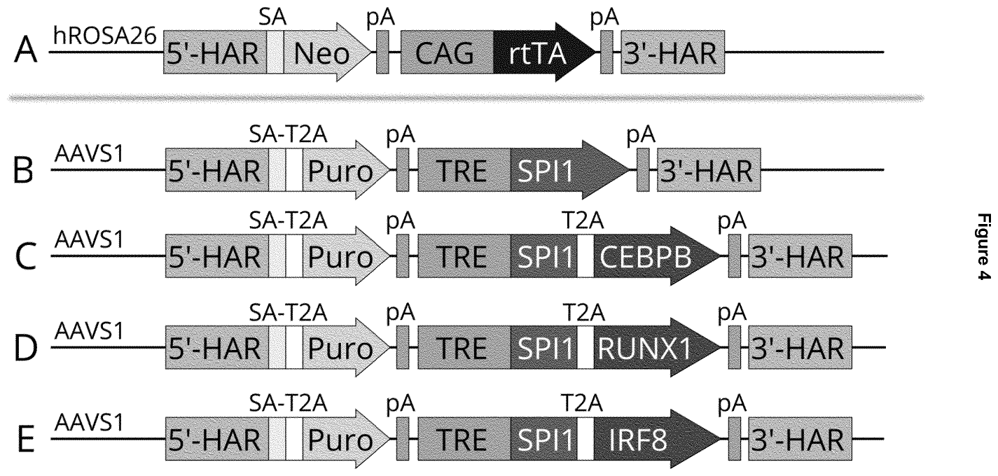

As used within the present invention, the term “Tet Responsive Element (TRE)” means a bacterial TetO sequence of 7 repeats of 19 bp separated by spacer sequences, together with a minimal promoter. Variants and modifications of the TRE sequence are possible, since the minimal promoter can be any suitable promoter. Preferably, the minimal promoter shows no or minimal expression levels in the absence of rtTA binding. The inducible promoter inserted into the second GSH may thus comprise a TRE. The basic genetic principal underlying the present invention is also depicted in FIG. 2, showing the different GSH sites (hROSA26 and AAVS1), and the integrated rtTA (SEQ ID NO: 20) and TRE (SEQ ID NO: 21).

In a further embodiment of the method of the present invention, said first and said second genomic safe harbour sites are selected from the group consisting of the hROSA26 locus (SEQ ID NO: 22), the AAVS1 locus (SEQ ID NO: 23), the CLYBL gene (SEQ ID NO: 24), the CCR5 gene (SEQ ID NO. 25), the HPRT gene (SEQ ID NO. 26) or genes with the site ID 325 on chromosome 8 (SEQ ID NO: 27), site ID 227 on chromosome 1 (SEQ ID NO: 28), site ID 229 on chromosome 2 (SEQ ID NO: 29), site ID 255 on chromosome 5 (SEQ ID NO: 30), site ID 259 on chromosome 14 (SEQ ID NO: 31), site ID 263 on chromosome X (SEQ ID NO: 32), site ID 303 on chromosome 2 (SEQ ID NO: 33), site ID 231 on chromosome 4 (SEQ ID NO: 34), site ID 315 on chromosome 5 (SEQ ID NO: 35), site ID 307 on chromosome 16 (SEQ ID NO: 36), site ID 285 on chromosome 6 (SEQ ID NO: 37), site ID 233 on chromosome 6 (SEQ ID NO: 38), site ID 311 on chromosome 134 (SEQ ID NO: 39), site ID 301 on chromosome 7 (SEQ ID NO: 40), site ID 293 on chromosome 8 (SEQ ID NO: 41), site ID 319 on chromosome 11 (SEQ ID NO: 42), site ID 329 on chromosome 12 (SEQ ID NO: 43), site ID 313 on chromosome X (SEQ ID NO: 44). Preferably, in a further embodiment of the method of the present invention, said first and said second genomic safe harbour sites are selected from the group consisting of the hROSA26 locus (SEQ ID NO: 22), the AAVS1 locus (SEQ ID NO: 23), the CLYBL gene (SEQ ID NO: 24), the CCR5 gene (SEQ ID NO. 25), the HPRT gene (SEQ ID NO. 26). More preferably, said first and said second genomic safe harbour sites are selected from the group consisting of the hROSA26 locus (SEQ ID NO: 22) and the AAVS1 locus (SEQ ID NO: 23).

Further sites may be identified by looking for sites where viruses naturally integrate without disrupting natural gene expression. For the method of the present invention, several GSH sites may be used, which will be described in more detail in the following.

The adeno-associated virus integration site 1 locus (AAVS1) (SEQ ID NO: 23) is located within the protein phosphatase 1, regulatory subunit 12C (PPP1R12C) gene on human chromosome 19, which is expressed uniformly and ubiquitously in human tissues. This site serves as a specific integration locus for AAV serotype 2, and thus was identified as a possible GSH. AAVS1 has been shown to be a favourable environment for transcription, since it comprises an open chromatin structure and native chromosomal insulators that enable resistance of the inducible cassettes against silencing. There are no known adverse effects on a cell resulting from disruption of the PPP1R12C gene. Moreover, an inducible cassette inserted into this site remains transcriptionally active in many diverse cell types. AAVS1 is thus considered to be a GSH and has been widely utilized for targeted transgenesis in the human genome.

The hROSA26 site (SEQ ID NO: 22) has been identified on the basis of sequence analogy with a GSH from mice (ROSA26—reverse oriented splice acceptor site #26). Although the orthologue site has been identified in humans, this site is not commonly used for inducible cassette insertion. The inventors of the present invention have used a targeting system specifically for the hROSA26 site and thus were able to insert genetic material into this locus. The hROSA26 locus (SEQ ID NO: 22) is on chromosome 3 (3p25.3), and can be found within the Ensembl database (GenBank: CR624523). The exact genomic co-ordinates of the integration site are 3:9396280-9396303: Ensembl. The integration site lies within the open reading frame (ORF) of the THUMPD3 long non-coding RNA (reverse strand). Since the hROSA26 site has an endogenous promoter, the inserted genetic material may take advantage of that endogenous promoter, or alternatively, may be inserted operably linked to a promoter.

Intron 2 of the Citrate Lyase Beta-like (CLYBL) gene (SEQ ID NO: 24), on the long arm of Chromosome 13, was identified as a suitable GSH since it is one of the identified integration hot-spots of the phage derived phiC31 integrase. Studies have demonstrated that randomly inserted inducible cassettes into this locus are stable and expressed. It has been shown that insertion of inducible cassettes at this GSH does not perturb local gene expression (Cerbibi et al., 2015). CLYBL thus provides a GSH which may be used in the method of the present invention.

CCR5 (SEQ ID NO: 25), which is located on chromosome 3 (position 3p21.31) is a gene, which codes for HIV-1 major co-receptor. Interest in the use of this site as a GSH arises from the null mutation in this gene that appears to have no adverse effects, but predisposes to HIV-1 infection resistance. Zinc-finger nucleases that target the third exon have been developed, thus allowing for insertion of genetic material at this locus. Given that the natural function of CCR5 has yet to be elucidated, the site remains a putative GSH, which may be used in the method of the present invention. The hypoxanthine-guanine phosphoribosyl transferase (HPRT) gene encodes a transferase enzyme that plays a central role in the generation of purine nucleotides through the purine salvage pathway. It has been mooted as a GSH site. Insertions at this site may be more applicable for mature cell types, such as modification for gene therapy. GSH in other organisms have been identified and include ROSA26, HRPT and HippII (HII) loci in mice.

Mammalian genomes may include GSH sites based upon pseudo attP sites. For such sites, hiC31 integrase, the Streptomyces phage-derived recombinase, has been developed as a non-viral insertion tool, because it has the ability to integrate an inducible cassette-containing plasmid carrying an attB site into pseudo attP sites. GSH are also present in the genomes of plants, and modification of plant cells can be used in the method of the present invention. GSH have been identified in the genomes of rice (Cantos et al., 2014).

The following SHS sites may be used in any of the methods of the present invention. They were published by Pellenz et al., 2019, and fulfil five out of nine criteria listed above: Site ID 325 on chromosome 8:68,720,172-68,720,191 (SEQ ID NO: 27); site ID 227 on chromosome 1:231,999,396-231,999,415 (SEQ ID NO: 28); site ID 229 on chromosome 2:45,708,354-45,708,373 (SEQ ID NO: 29); site ID 255 on chromosome 5:19,069,307-19,069,326 (SEQ ID NO: 30); site ID 259 on chromosome 14:92,099,558-92,099,577 (SEQ ID NO: 31); site ID 263 on chromosome X:12,590,812-12,590,831 (SEQ ID NO: 32); site ID 303 on chromosome 2:77,263,930-77,263,949 (SEQ ID NO: 33); site ID 317 on chromosome 2:77,263,930-77,263,949 (SEQ ID NO: 60); site ID 231 on chromosome 4:58,976,613-58,976,632 (SEQ ID NO: 34); site ID 315 on chromosome 5:7,577,728-7,577,747 (SEQ ID NO: 35); site ID 307 on chromosome 16:19,323,777-19,323,796 (SEQ ID NO: 36); site ID 285 on chromosome 6:89,574,320-89,574,339 (SEQ ID NO: 37); site ID 233 on chromosome 6:114,713,905-114,713,924 (SEQ ID NO: 38); site ID 311 on chromosome 6:134,385,946-134,385,965 (SEQ ID NO: 39); site ID 301 on chromosome 7:113,327,685-113,327,704 (SEQ ID NO: 40); site ID 293 on chromosome 8:40,727,927-40,727,946 (SEQ ID NO: 41); site ID 319 on chromosome 11:32,680,546-32,680,565 (SEQ ID NO: 42); site ID 329 on chromosome 12:126,152,581-126,152,600 (SEQ ID NO: 43); and site ID 313 on chromosome X:16,059,732-16,059,751 (SEQ ID NO: 44).

In another embodiment of the method of the present invention, said stem cell is a pluripotent stem cell, an induced pluripotent stem cell (iPSC), a neural progenitor cell, hematopoietic stem cell or an embryonic stem cell (ESC).

Within the present invention, the term “pluripotent stem cell” is used as defined above.

As used within the present invention, the term “neural progenitor cell” means a multipotent cell state between pluripotent stem cell and mature somatic cell. This cell state is usually determined to become a specialized cell type like neurons, oligodendrocytes and astrocytes.

Within the present invention, the term “induced pluripotent stem cell (iPSC)” is used as defined above.

As used within the present invention, the term “hematopoietic stem cell” means a blood forming stem cell. This special type of multipotent stem cell is able to form any type of blood cell, but lost the capacity to form other cell types.

Within the present invention, the term “embryonic stem cell (ESC)” is used as defined above.

In a further embodiment of the method of the present invention, said stem cell is a human or a mouse stem cell.

As used within the present invention, the term “human or a mouse stem cell” means a cell originated from human or mouse. However, the stem cell used in the method of the present invention may be any human or animal cell. It is preferably a mammalian cell, such as a cell from a rodent, such as mice and rats; marsupial such as kangaroos and koalas; non-human primate such as a bonobo, chimpanzee, lemurs, gibbons and apes; camelids such as camels and llamas; livestock animals such as horses, pigs, cattle, buffalo, bison, goats, sheep, deer, reindeer, donkeys, bantengs, yaks, chickens, ducks and turkeys; domestic animals, such as cats, dogs, rabbits and guinea pigs. The cell is preferably a human cell. In certain aspects, the cell is preferably one from a livestock animal. The type of cell used in the method of the present invention will depend upon the application of the cell once insertion of the genetic material into the GSH sites is complete.

The present invention also relates to a microglia cell obtained by any of the methods according to the present invention, preferably wherein the microglia expresses at least one microglia surface protein selected from the group consisting of ITGAM (CD11B) (SEQ ID NO: 45), ITGAX (CD11C) (SEQ ID NO: 46), CD14 (SEQ ID NO: 47), CD16 (SEQ ID NO: 48), ENTPD1 (CD39) (SEQ ID NO: 49), PTPRC (CD45) (SEQ ID NO: 50), CD68 (SEQ ID NO: 51), CSF1R (CD115) (SEQ ID NO: 52), CD163 (SEQ ID NO: 53), CX3CR1 (SEQ ID NO: 54), TREM2 (SEQ ID NO: 55), P2RY12 (SEQ ID NO: 56), TMEM119 (SEQ ID NO: 57), and HLA-DR (SEQ ID NO: 58).

Thus, microglia are additionally defined by expressing at least one of the following surface proteins ITGAM (CD11B) (SEQ ID NO: 45), ITGAX (CD11C) (SEQ ID NO: 46), CD14 (SEQ ID NO: 47), CD16 (SEQ ID NO: 48), ENTPD1 (CD39) (SEQ ID NO: 49), PTPRC (CD45) (SEQ ID NO: 50), CD68 (SEQ ID NO: 51), CSF1R (CD115) (SEQ ID NO: 52), CD163 (SEQ ID NO: 53), CX3CR1 (SEQ ID NO: 54), TREM2 (SEQ ID NO: 55), P2RY12 (SEQ ID NO: 56), TMEM119 (SEQ ID NO: 57), and HLA-DR (SEQ ID NO: 58). These proteins are defined as follows.

ITGAM (CD11B), as used in the present invention, means Integrin Subunit Alpha M, a gene which encodes the integrin alpha M chain. Integrins are heterodimeric integral membrane proteins composed of an alpha chain and a beta chain. The protein sequence thereof is given in SEQ ID NO: 45.

ITGAX (CD11C), as used within the present invention, means Integrin Subunit Alpha X, and the gene encodes the integrin alpha X chain protein. The protein sequence thereof is given in SEQ ID NO: 46.

CD14, as used in the present invention, means Monocyte Differentiation Antigen CD14 and the protein encoded by this gene is a surface antigen that is preferentially expressed on monocytes/macrophages. The protein sequence thereof is given in SEQ ID NO: 47.

CD16, as used in the present invention, means FCGR3A Fc Fragment of IgG Receptor IIIa and this gene encodes a receptor for the Fc portion of immunoglobulin G, and it is involved in the removal of antigen-antibody complexes from the circulation, as well as other antibody-dependent responses. The protein sequence thereof is given in SEQ ID NO: 48.

ENTPD1 (CD39), as used in the present invention, means Ectonucleoside Triphosphate Diphosphohydrolase 1 and the protein encoded by this gene is a plasma membrane protein that hydrolyzes extracellular ATP and ADP to AMP. The protein sequence thereof is given in SEQ ID NO: 49.

PTPRC (CD45), as used in the present invention, means Protein Tyrosine Phosphatase Receptor Type C and the protein encoded by this gene is a member of the protein tyrosine phosphatase (PTP) family. The protein sequence thereof is given in SEQ ID NO: 50.

CD68, as used in the present invention, means CD68 Antigen and this gene encodes a 110-kD transmembrane glycoprotein that is highly expressed by human monocytes and tissue macrophages. The protein sequence thereof is given in SEQ ID NO: 51.

CSF1R (CD115), as used in the present invention, means Colony Stimulating Factor 1 Receptor and the protein encoded by this gene is the receptor for colony stimulating factor 1, a cytokine which controls the production, differentiation, and function of macrophages. The protein sequence thereof is given in SEQ ID NO: 52.

CD163, as used in the present invention, means CD163 Antigen and the protein encoded by this gene is a member of the scavenger receptor cysteine-rich (SRCR) superfamily, and is exclusively expressed in monocytes and macrophages. The protein sequence thereof is given in SEQ ID NO: 53.

CX3CR1, as used in the present invention, means C-X3-C Motif Chemokine Receptor 1 and the protein encoded by this gene is a receptor for fractalkine. The protein sequence thereof is given in SEQ ID NO: 54. Fractalkine is a transmembrane protein and chemokine involved in the adhesion and migration of leukocytes.

TREM2, as used in the present invention, means Triggering Receptor Expressed On Myeloid Cells 2 and this gene encodes a membrane protein that forms a receptor signaling complex with the TYRO protein tyrosine kinase binding protein. The protein sequence thereof is given in SEQ ID NO: 55.

P2RY12, as used in the present invention, means Purinergic Receptor P2Y12 and the product of this gene belongs to the family of G-protein coupled receptors. The protein sequence thereof is given in SEQ ID NO: 56.

TMEM119, as used in the present invention, means Transmembrane Protein 119, which is a protein coding gene. Among its related pathways are microglia activation during neuroinflammation. The protein sequence thereof is given in SEQ ID NO: 57.

HLA-DR, as used in the present invention, means Major Histocompatibility Complex, Class II, DR Alpha and Beta and both HLA-DRA and HLA-DRB1 are HLA class II alpha chain paralogues. The protein sequence thereof is given in SEQ ID NO: 58.

In a further embodiment, the present invention also comprises the microglia cell according to the present invention for use in therapy.

As used in the present invention, the term “therapy” means any form of treatment of diseases or unwanted health status of organisms, animals or human beings. It may also include gene therapy. This may be defined as the intentional insertion of foreign DNA into the nucleus of a cell with therapeutic intent. Such a definition includes the provision of a gene or genes to a cell to provide a wild type version of a faulty gene, the addition of genes for RNA molecules that interfere with target gene expression (which may be defective), provision of suicide genes (such as the enzymes herpes simplex virus, thymidine kinase (HSV-tk) and cytosine deaminase (CD), which convert the harmless prodrug ganciclovir (GCV) into a cytotoxic drug, DNA vaccines for immunization or cancer therapy (including cellular adoptive immunotherapy) and any other provision of genes to a cell for therapeutic purposes. Additionally, the mature microglia may be used directly for transplantation into a human or animal body. Alternatively, the microglia may form a test material for research, including the effects of drugs on gene expression and the interaction of drugs with a particular gene. The microglia for research can involve the use of an inducible cassette with a genetic sequence of unknown function, in order to study the controllable expression of that genetic sequence. Additionally, it may enable the microglia to be used to produce large quantities of desirable materials, such as growth factors or cytokines.

Further, the present invention is also directed in one embodiment to the use of such a microglia cell according to the present invention for in vitro diagnostics of a disease. Preferably, the disease is selected from the group consisting of diseases of the central nervous system, preferably neurodegenerative diseases; more preferably Alzheimer's disease, Parkinson's disease, frontotemporal dementia or Amyotrophic Lateral Sclerosis; neuroinflammatory or autoimmune diseases, preferably Multiple Sclerosis, auto-antibody-mediated encephalitis or infectious diseases, neurovascular diseases; preferably stroke, vasculitis; traumatic brain injury, and cancer.

Further, the present invention is directed to the use of such a microglia cell according to the present invention for in vitro culturing with brain organoids.

As used within the present invention, the term “organoid” means (mostly stem) cell-derived in vitro 3D-organ models and represent in combination with the microglia produced according to this invention a powerful tool for medical diagnostics to study the involvement and interaction of microglia with other cells of the brain.

It is noted that as used herein, the singular forms “a”, “an”, and “the”, include plural references unless the context clearly indicates otherwise. Thus, for example, reference to “a reagent” includes one or more of such different reagents and reference to “the method” includes reference to equivalent steps and methods known to those of ordinary skill in the art that could be modified or substituted for the methods described herein.

Unless otherwise indicated, the term “at least” preceding a series of elements is to be understood to refer to every element in the series. The term “at least one” refers, if not particularly defined differently, to one or more such as two, three, four, five, six, seven, eight, nine, ten or more. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

The term “less than” or in turn “more than” does not include the concrete number.

For example, less than 20 mean less than the number indicated. Similarly, “more than” or “greater than” means more than or greater than the indicated number, e.g. more than 80% means more than or greater than the indicated number of 80%.

Throughout this specification and the claims which follow, unless the context requires otherwise, the word “comprise”, and variations such as “comprises” and “comprising”, will be understood to imply the inclusion of a stated integer or step or group of integers or steps, but not the exclusion of any other integer or step or group of integer or step. When used herein the term “comprising” can be substituted with the term “containing” or “including” or sometimes when used herein with the term “having”. When used herein “consisting of” excludes any element, step, or ingredient not specified.

The term “including” means “including but not limited to”. “Including” and “including but not limited to” are used interchangeably.

The term “about” means plus or minus 10%, preferably plus or minus 5%, more preferably plus or minus 2%, most preferably plus or minus 1%.

Throughout the description and claims of this specification, the singular encompasses the plural unless the context otherwise requires. In particular, where the indefinite article is used, the specification is to be understood as contemplating plurality as well as singularity, unless the context requires otherwise.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

All publications cited throughout the text of this specification (including all patents, patent application, scientific publications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The content of all documents and patent documents cited herein is incorporated by reference in their entirety.

A better understanding of the present invention and of its advantages will be gained from the following examples, offered for illustrative purposes only. The examples are not intended to limit the scope of the present invention in any way.

### EXAMPLES OF THE INVENTION

The following Examples illustrate the invention, but are not to be construed as limiting the scope of the invention.

### Example 1

Material and Methods

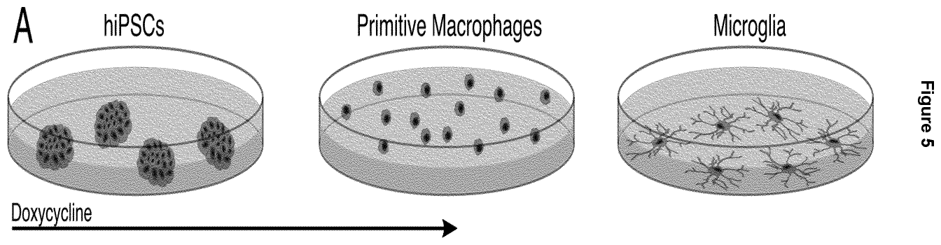

For initial screening experiments, first an hROSA-CAG-rtTA hiPSC-line (nucleofection of three plasmids expressing a CAS9-nickase, two hROSA26-specific guideRNAs (SEQ ID NO: 66 and SEQ ID NO: 67) and the donor plasmid with the CAG-rtTA expression cassette as demonstrated in FIG. 2A and FIG. 4A; antibiotic selection, clonal expansion and characterization of individual clonal hiPSC-colonies) was generated and subsequent transient transfection of the four AAVS1 targeting vectors (SEQ ID NOs: 61 to SEQ ID NO: 64) (see also FIG. 4B-E) was performed allowing for quick overexpression of PU.1 (SEQ ID NO: 2) either alone or in combination with either of the three other transcription factors RUNX1 (SEQ ID NO: 4), CEBPB (SEQ ID NO: 3), or IRF8 (SEQ ID NO: 5) in the form of a bi-cistronic expression cassette (FIG. 4B-E) (SEQ ID NO: 61 to SEQ ID NO. 64). For screening purpose, targeted cells were not clonally expanded, resulting in overexpression only in a subset of cells.

Surprisingly, initial screening experiments demonstrated rapid induction of myeloid and microglia lineage marker in all three cell lines expressing PU.1 (SEQ ID NO: 2) plus any of the other three candidate reprogramming factors, but not in wild-type control hiPSCs or in cells expressing PU.1 (SEQ ID NO: 2) alone.

Description

To develop a prototype protocol and establish suitable read-out parameters, the inventors decided to focus on the combinatorial overexpression of PU.1 (SEQ ID NO: 2) and CEBPB (SEQ ID NO: 3). Thus, fully verified dual GSH targeted inducible PU.1+CEBPB hiPSCs were created and clonally expanded.

Observation

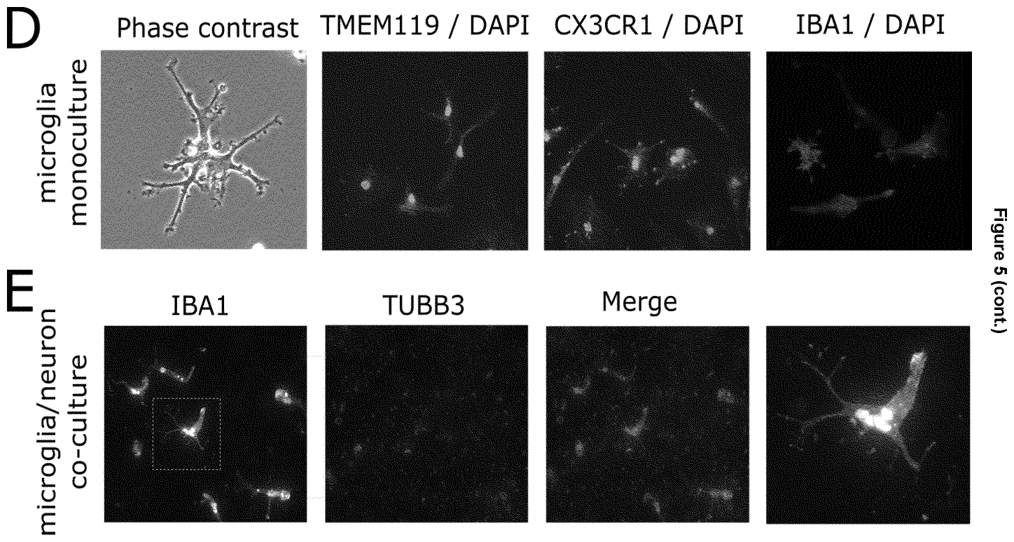

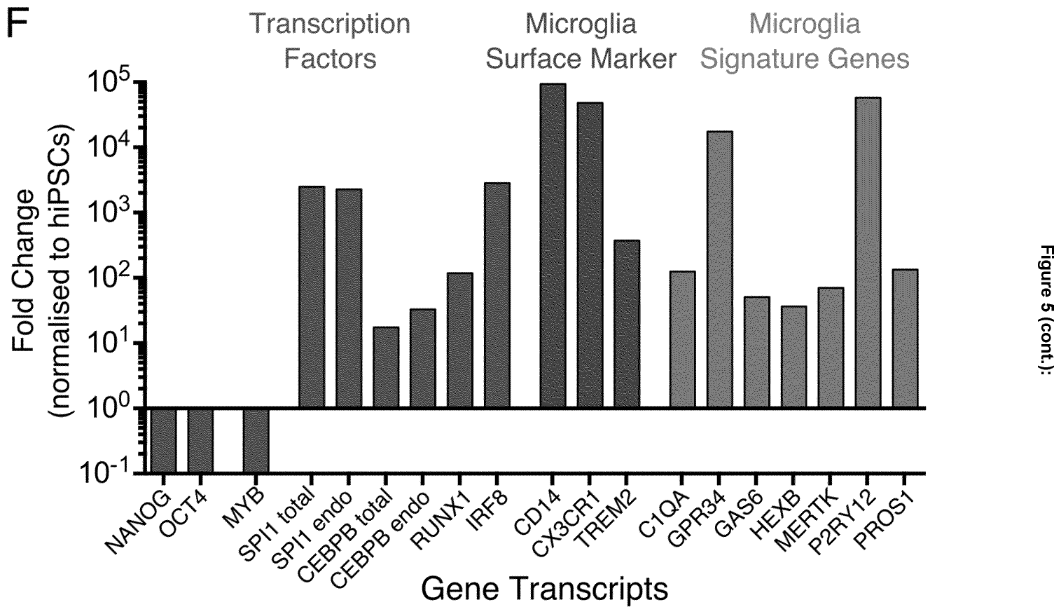

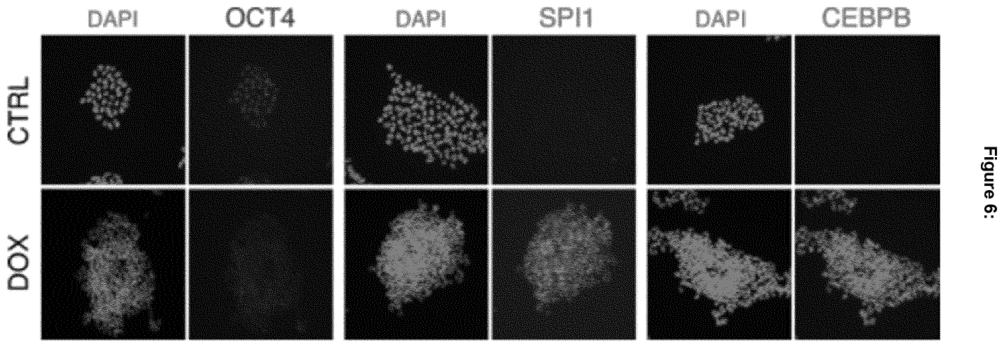

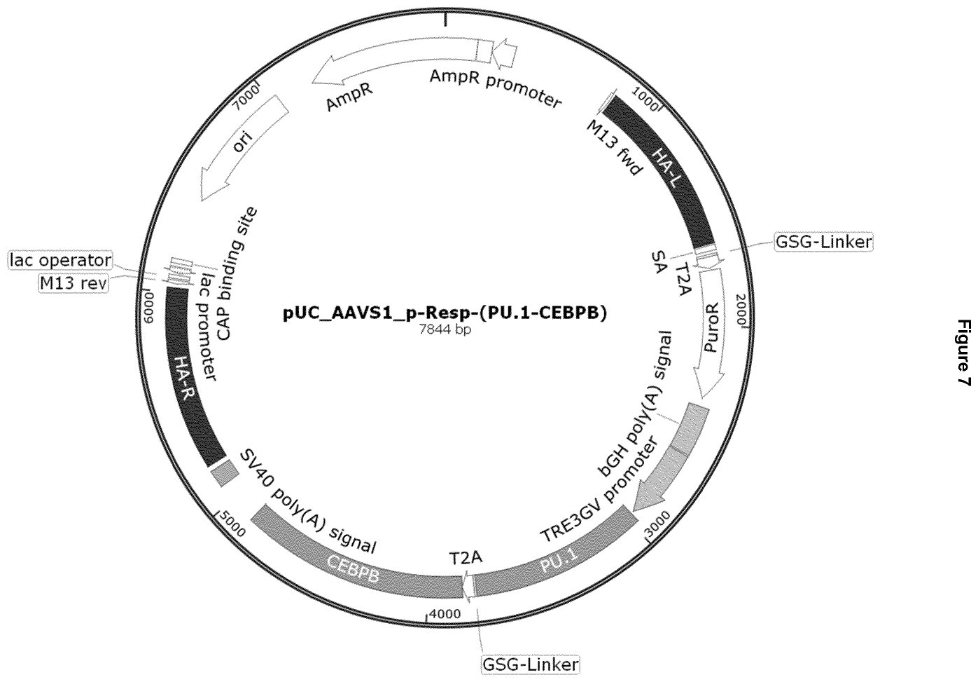

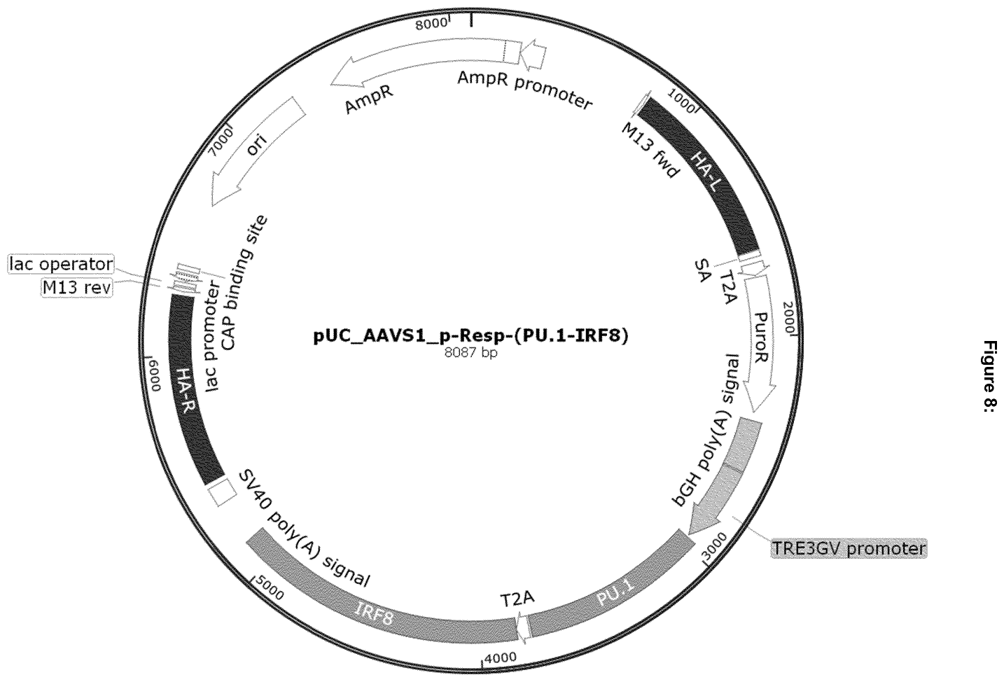

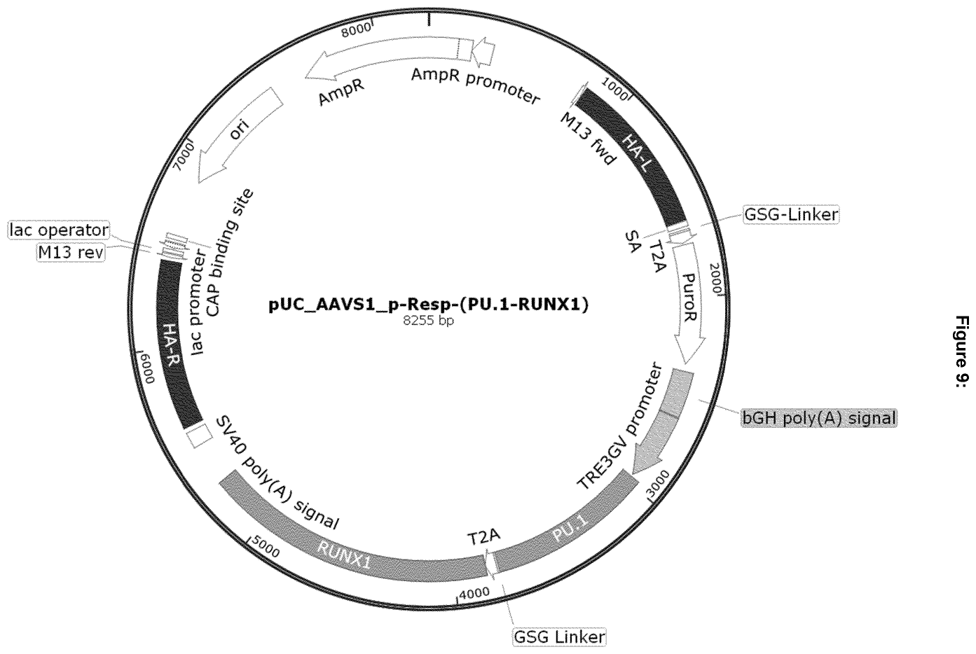

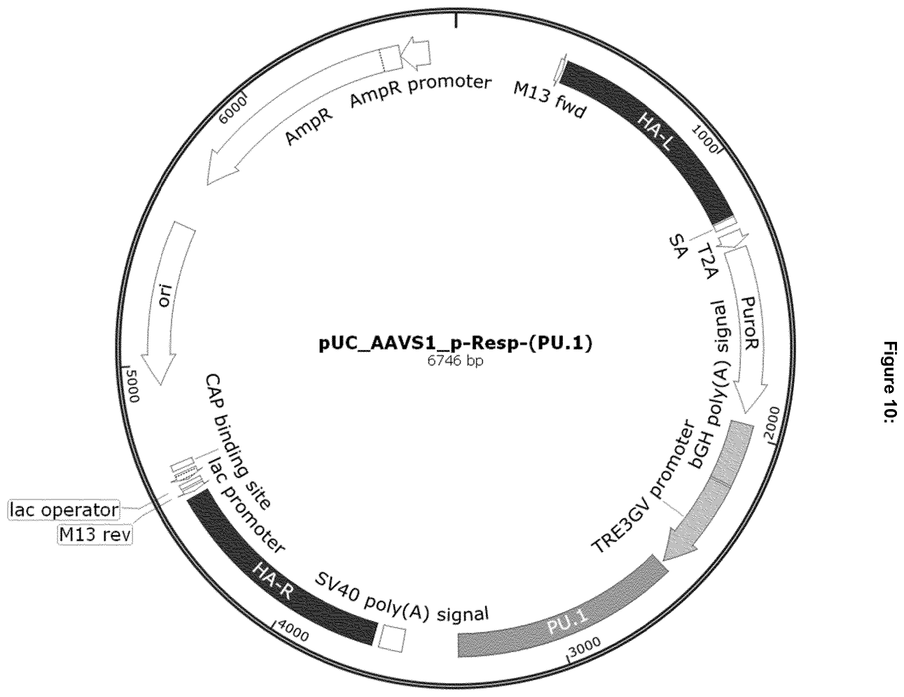

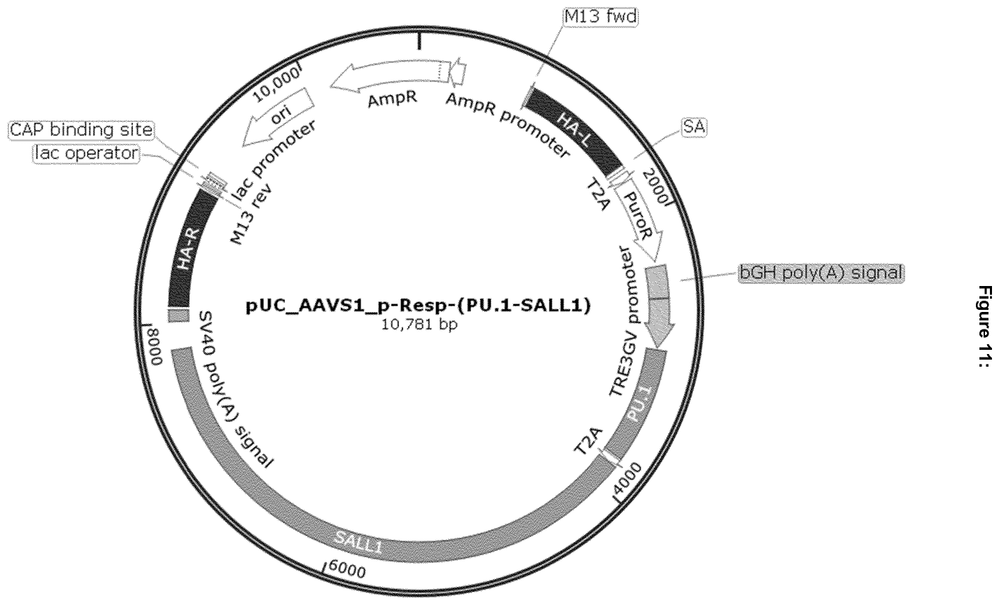

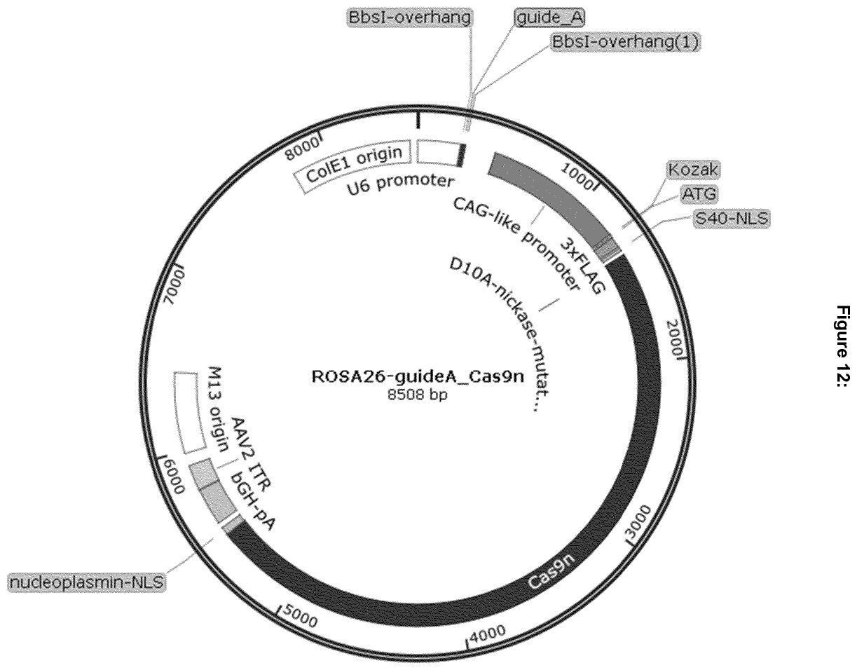

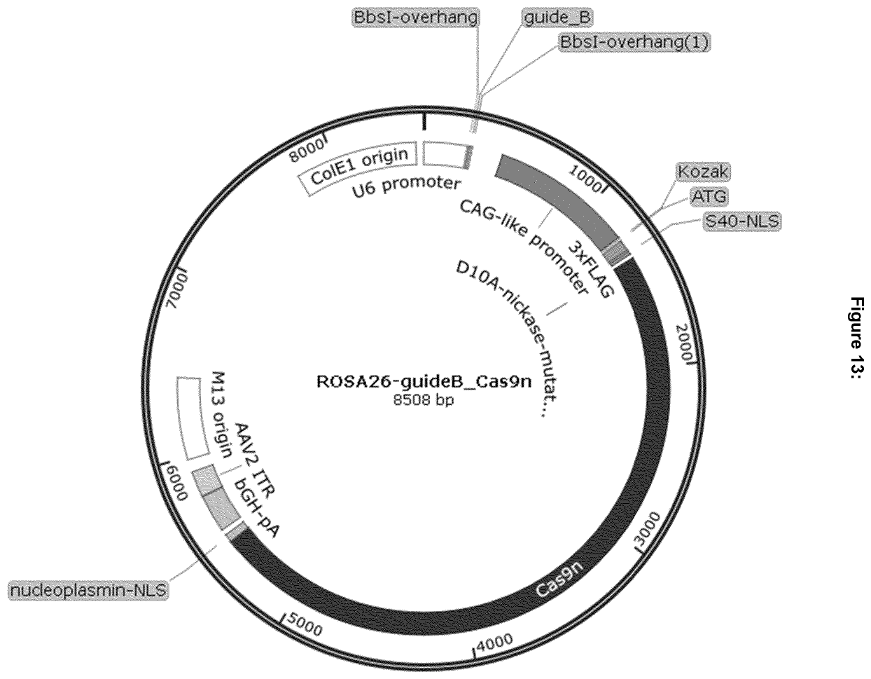

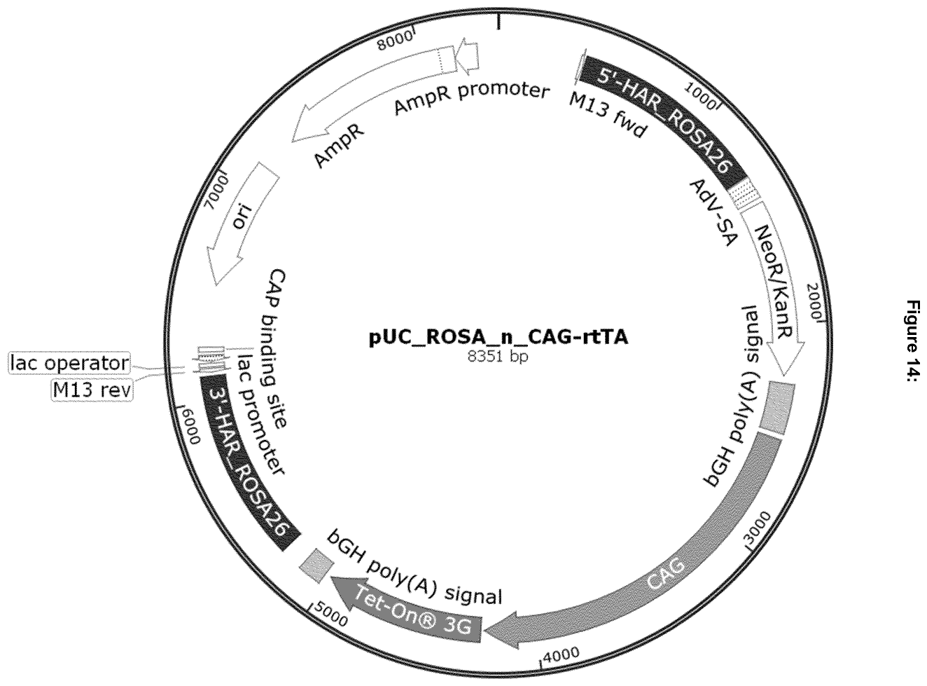

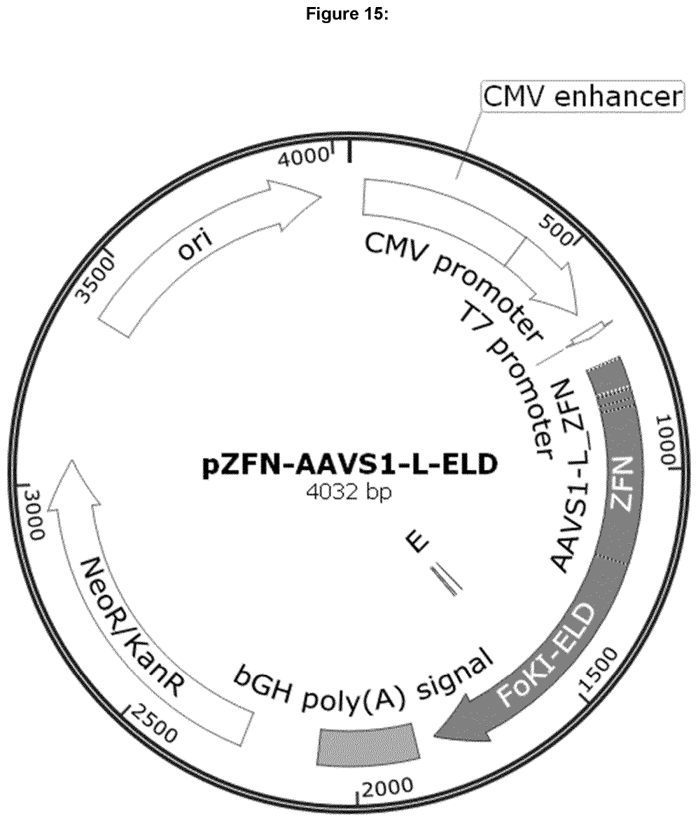

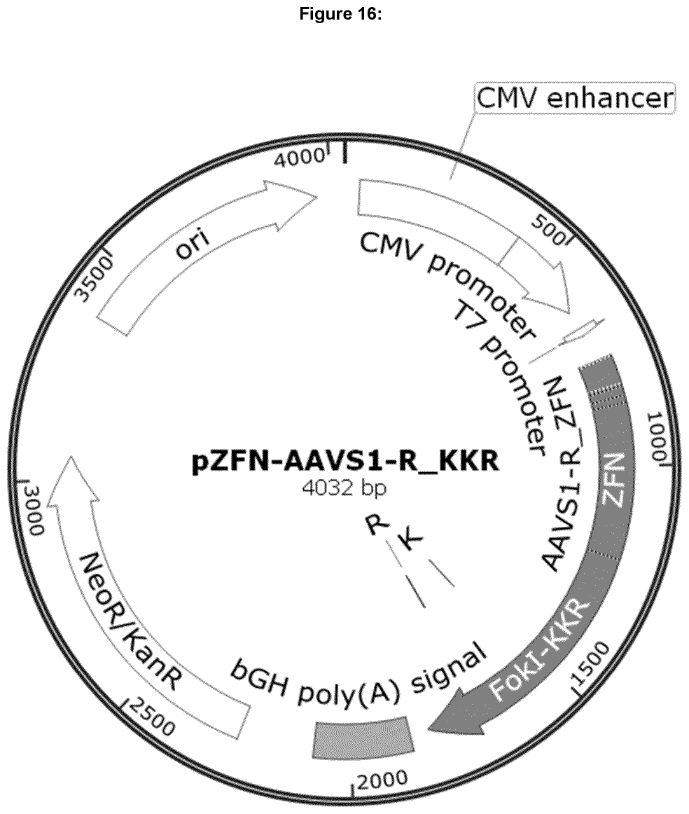

Addition of doxycycline resulted in the rapid loss of expression of the pluripotency markers OCT4 (SEQ ID NO: 78) and NANOG (SEQ ID NO: 79) and induction of both transgenes in all cells (see FIG. 6).

### Example 2

Material and Methods

In brief, hiPSCs were plated as single cells onto Matrigel in pluripotency maintenance medium. After two days, the media is changed to Dulbecco's modified eagle medium (DMEM)/F12 supplemented with dox for transgene induction plus small molecules and growth factors mimicking the sequence of embryonic events outlined above. After three days of induction, the adherent cells started to delaminate from the tissue culture plate and were found as floating single cells in the supernatant.

Description

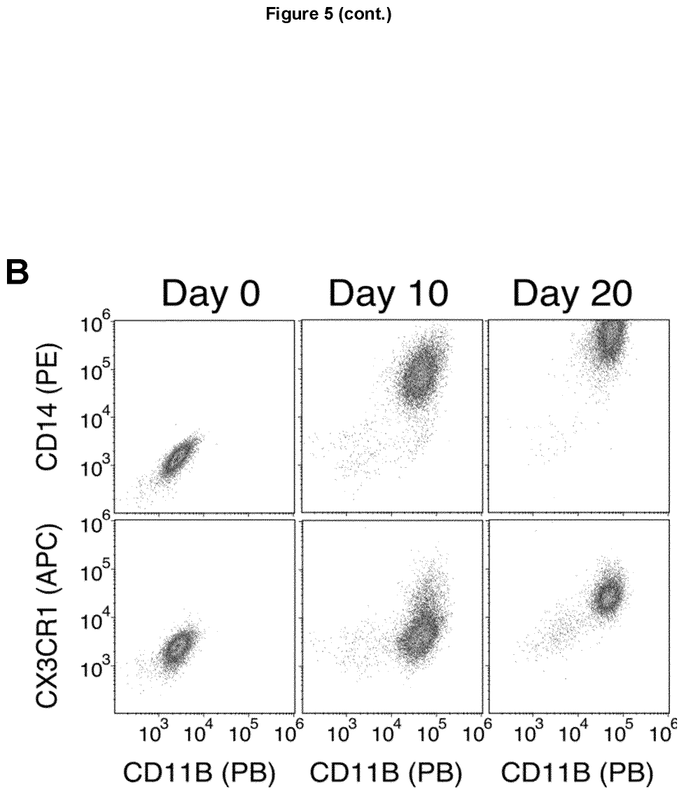

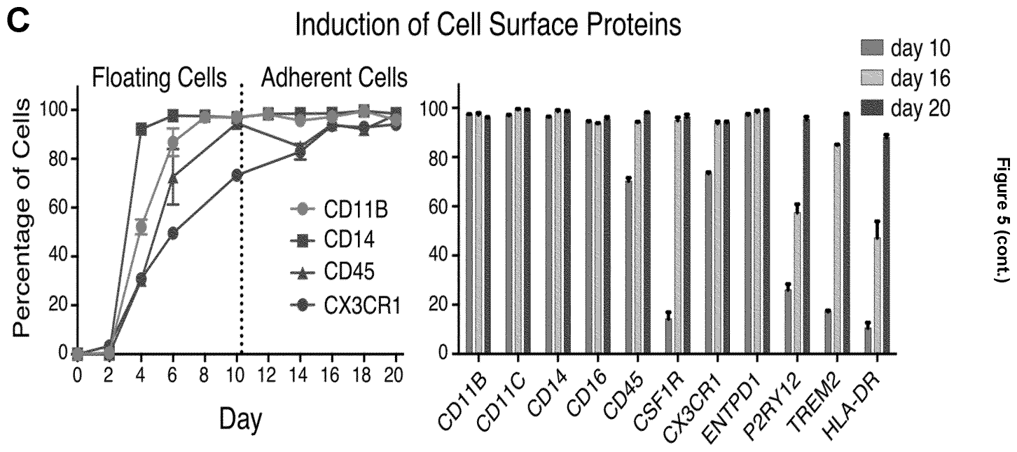

Subsequently, the inventors performed longer screening experiments in which cells were induced for up to 20 days for optimisation of media compositions. Multi-colour flow cytometry demonstrated a remarkably robust and rapid induction of myeloid cell surface markers that were chosen as screening panel for the induction of primitive macrophages and/or microglia (CD11b (SEQ ID NO: 45), CD14 (SEQ ID NO: 47), CD45 (SEQ ID NO: 50), CD163 (SEQ ID NO: 53), CX3CR1 (SEQ ID NO: 54)). The inventors also noted important culture condition-dependent differences: Induction occurred most rapidly and efficiently when the transcription factor overexpression was performed in conjunction with timed exposure to extracellular cues (small molecules, growth factors) mimicking the sequence of embryonic development: (1) patterning of the pluripotent epiblast (hiPSCs) towards (posterior primitive streak) extra-embryonic mesoderm and the haemangioblast, (2) induction of primitive haematopoiesis and early macrophage precursors, (3) differentiation into primitive yolk sac macrophages, (4) differentiation into microglia (see FIG. 5).

Observation

The cells rapidly started to express typical myeloid surface proteins including CD45 (SEQ ID NO: 50) (also known as PTPRC), CD11b (SEQ ID NO: 45) (also known as ITGAM), CD14 (SEQ ID NO: 47), and CX3CR1 (SEQ ID NO: 54) as demonstrated by flow cytometry (see FIG. 5B-C). By day 10, all cells had transitioned into the supernatant and were plated down onto poly-L-lysine (PLL) coated tissue culture dishes in final, chemically-defined microglia differentiation and maintenance medium, according to Muffat et al., 2016. Interestingly, differentiation into microglia occurred even more efficiently when doxycycline was withdrawn after day ten of the induction protocol, thus unequivocally demonstrating the independence of the cellular phenotype from continued transgene expression.

After 6-10 days of transgene-free differentiation and maturation in adhesion culture, virtually all cells expressed a wide range of common myeloid and more microglia specific proteins, including CD39 (SEQ ID NO: 49), P2RY12 (SEQ ID NO: 56), TREM2 (SEQ ID NO: 55), and TMEM119 (SEQ ID NO: 57) as quantified by flow cytometry (see FIG. 5c) or demonstrated by immunocytochemistry (see FIG. 5D). Next, co-culture experiments were performed in which the inventors plated microglia precursors onto a pure population of isogenic hiPSC-derived cortical neurons generated according to previously published protocol according to Zhang et al., 2013, and Pawlowski et al., 2017. Microglial cells acquired a more ramified (i.e. less activated) morphology compared to cells in monoculture (see FIG. 5E). Real-time qPCR analysis of hiPSCs and microglia in monoculture demonstrated downregulation of pluripotency factors, MYB-independence (in line with the primitive yolk sac macrophage origin of microglia), and high expression of core microglia transcription factors, classical surface markers, and recently suggested unique microglial signature genes (see FIG. 5F).

### REFERENCES

Abud E M, Ramirez R N, Martinez E S, Healy L M, Nguyen C H H, Newman S A, Yeromin A V, Scarfone V M, Marsh S E, Fimbres C, et al.: iPSC-Derived Human Microglia-like Cells to Study Neurological Diseases. Neuron, 2017, 94:278-293.e9.
Butovsky O, Weiner H L: Microglial signatures and their role in health and disease. Nat Rev Neurosci., 2018, 19.
Cantos C, Francisco P, Trijatmiko K R, Slamet-Loedin I and Chadha-Mohanty P K: Identification of “safe harbor” loci in indica rice genome by harnessing the property of zinc-finger nucleases to induce DNA damage and repair. Frontier in Plant Science, 2014, 5: 302, pp. 1-8.
Cerbini T, Funahashi R, Luo Y, Liu C, Park K, Rao M, Malik N, Zou J: Transcription Activator-Like Effector Nuclease (TALEN)-Mediated CLYBL Targeting Enables Enhanced Transgene Expression and One-Step Generation of Dual Reporter Human Induced Pluripotent Stem Cell (iPSC) and Neural Stem Cell (NSC) Lines. Plos One, 2015.
Chung Y, Klimanskaya I, Becker S, Li T, Maserati M, Lu S, Zdravkovic T, Ilic D, Genbacev O, Fisher S, Krtolica A, and Lanza R: Human Embryonic Stem Cell Lines Generated without Embryo Destruction. 2008, Cell Stem Cell, 2(2) pp. 113-117.
Cohen D E, Melton D A: Turning straw into gold: directing cell fate for regenerative medicine. Nat Rev Genet 2011, 12:243-52.
Douvaras P, Sun B, Wang M, Kruglikov I, Lallos G, Zimmer M, Terrenoire C, Zhang B, Gandy S, Schadt E, et al.: Directed Differentiation of Human Pluripotent Stem Cells to Microglia. Stem cell reports, 2017, 8:1516-1524.
Gaj, T, Gersbach, C A, Barbas, C F: ZFN, TALEN, and CRISPR/Cas-based methods for genome engineering, Trends Biotechnol., 2013, 31(7):397-405.
Ginhoux F, Greter M, Leboeuf M, Nandi S, See P, Gokhan S, Mehler M F, Conway S J, Ng L G, Stanley E R, et al.: Fate mapping analysis reveals that adult microglia derive from primitive macrophages. Science, (80) 2010, 330:841-5.
Gomez Perdiguero E, Klapproth K, Schulz c, Busch K, Azzoni E, Crozet L, Garner H, Trouillet C, de Bruijn M F, Geissmann F, et al.: Tissueresident macrophages originate from yolk-sac-derived erythro-myeloid progenitors. Nature 2015, 518:547-51.
Haenseler W, Sansom S N, Buchrieser J, Newey S E, Moore C S, Nicholls F J, Chintawar S, Schnell C, Antel J P, Allen N D, et al.: A Highly Efficient Human Pluripotent Stem Cell Microglia Model Displays a Neuronal-Co-culture-Specific Expression Profile and inflammatory Response. Stem cell reports 2017, 8:1727-1742.
Hagan C E, Bolon B and Keene C D, Comparative Anatomy and Histology, 2012, pages 339-394.
Keren-Shaul H, Spinrad A, Weiner A, Matcovitch-Natan O, Dvir-Szternfeld R, Ulland T K, David E, Baruch K, Lara-Astaiso D, Toth B, et al.: A unique Microglia Type Associated with Restricting Development of Alzheimer's Disease. Cell, 2017, 169:1276-1290.e17.
Kettenmann H, Hanisch U-K, Noda M, Verkhralsky A: Physiology of microglia. Physiol Rev., 2011, 91:461-553.
Krasemann S, Madore C, Cialic R, Baufeld C, Calcagno N, El Fatimy R, Beckers L, O'Loughlin E, Xu Y, Fanek Z, et al.: The TREM2-APOE Pathway Drives the Transcriptional Phenotype of Dysfunctional Microglia in Neurodegenerative Diseases. Immunity, 2017, 17:566581.e9.
Ladewig J, Koch P, Brüstle O: Leveling Waddington: the emergence of direct programming and the loss of cell fate hierarchies. Nat Rev Mol Cell Biol, 2013, 14:225-36.
McGrath K E, Koniski A D, Malik J, Palis J: Circulation is established In a stepwise pattern in the mammalian embryo. Blood, 2003, 101:1669-76.
Muffat J, Li Y, Yuan B, Mitalipova M, Omer A, Corcoran S, Bakiasi c, Tsai L-H, Aubourg P, Ransohoff R M, et al.: Efficient derivation of microglia-like cells from human pluripotent stem cells. Nat Med, 2016, 22:1355-1367.
Pandya H, Shen M J, Ichikawa D M, Sedlock A B, Choi Y, Johnson K R, Kim G, Brown M A, Elkahloun A G, Maric D, et al.: Differentiation of human and murine induced pluripotent stem cells to microglia-like cells. Nat Neurosci, 2017, 20:753-759.
Pawlowski M, Ortmann D, Bertero A, Tava.es J M, Pedersen R A, Vallier L, Kotter M R N: Inducible and Deterministic Forward Programming of Human Pluripotent Stem Cells into Neurons, Skeletal Myocytes, and Oligodendrocytes. Stem cell reports, 2017, 8:80H12.
Pellenz S, Phelps M, Tang W, Hovde T B, Sinit R B, Fu W, Li H, Chen E and Monnat, Jr. R J: New human chromosomal sites with ‘safe harbor’ potential for targeted transgene insertion. Human Gene Therapy, 2019, 1-47.
Qi Hui, Zi Jin, Xiaokun Li, Changxiao Liu and Xiaojie Wang: FGF Family: From Drug Development to Clinical Application. Int. J. Mol. Sci., 2018, 19(7), 1875.
Ransohoff R M: How neuroinflammation contributes to neurodegeneration. Science, (80-) 2016, 353:777-83.
Réu P, Khosravi A, Bernard S, Mold J E, Salehpour M, Alkass K, Perl S, Tisdale J, Possnert G, Druid H, et al.: The Lifespan and Turnover of Microglia in the Human Brain. Cell Rep, 2017, 20:77V784.
Schafer D P, Stevens B: Microglia Function in Central Nervous System Development and Plasticity. Cold Spring Harb Perspect Biol, 2015, 7:a020545.
Takahashi K, Tanabe K, Ohnuki M, Narita M, Ichisaka T, Tomoda K, Yamanaka S: Induction of pluripotent stem cells from adult human fibroblasts by defined factors. Cell, 2007, 131:861-72.
Takata K, Kozaki T, Lee C Z W, Thion M S, Otsuka M, Lim S, Utami K H, Fidan K, Park D S, Malleret B, et al.: Induced-Pluripotent-Stem-Cell-Derived Primitive Macrophages Provide a Platform for Modelling Tissue-Resident Macrophage Differentiation and Function. Immunity, 2017, 47: 1 83-1 98.e6.
Thomson J A, Itskovitz-Eldor J, Shapiro S S, Waknitz M A, Swiergiel J J, Marshall V S, Jones J M: Embryonic stem cell lines derived from human blastocysts. Science, (80-) 1998, 282:1145-7.
Vlahos C J, Matter W F, Hui K Y, and Brown R F: A Specific Inhibitor of Phosphatidylinositol 3-Kinase,2-(4-Morpholinyl)-8-phenyl-4H-l-benzopyran-4-one (LY294002). The Journal of Biological Chemistry, 1994, 269(7), pp. 5241-5248.
Zhang Y, Pak C, Han Y, Ahlenius H, ZhangZ, Chanda S, Marro S, Patzke C, Acuna c, Covy J, et al.: Rapid single-step induction of functional neurons from human pluripotent stem cells. Neuron, 2013, 78:785-98.

1. A method for the production of: microglia from stem cells, comprising the steps of:
a) targeted insertion of a nucleotide sequence encoding a transcriptional regulator protein into a first genomic safe harbour site; and
b) targeted insertion of the coding sequence of the transcription factor PU.1 (SEQ ID NO: 1) into a second genomic safe harbour site, wherein the gene is operably linked to an inducible promoter, which is regulated by the transcriptional regulator protein; expression of PU.1 (SEQ ID NO: 2); and
c) culturing the stem cells received from steps a) and b) with exposure to at least one growth factor or small molecule that recapitulates signaling during at least one stage of embryonic development of microglia or adult microglia proliferation, differentiation or polarization.

2. Method according to claim 1, wherein the at least one growth factor or small molecule is selected from the group consisting of Activin A (SEQ ID NO: 7), BMP4 (SEQ ID NO: 8), FGF (SEQ ID NO: 9), VEGF-A (SEQ ID NO: 10), LY294002, CHIR99021, SCF (SEQ ID NO: 11), IL-3 (SEQ ID NO: 12), IL-6 (SEQ ID NO: 13), CSF1 (SEQ ID NO: 14), IL-34 (SEQ ID NO: 15), CSF2 (SEQ ID NO: 16), CD200 (SEQ ID NO: 17), CX3CL1 (SEQ ID NO: 18), TGFβ1 (SEQ ID NO: 19), and IDE1.

3. Method according to claim 1 or 2, wherein the at least one growth factor is CSF1 (SEQ ID NO: 14) or IL-34 (SEQ ID NO: 15).

4. Method according to any one of the previous claims, wherein the at least one small molecule is CHIR99021, LY294002 or IDE1.

5. Method according to any one of the previous claims, wherein the first and the second genomic safe harbour sites are different.

6. Method of any one of the previous claims, further comprising insertion of the coding sequence of the gene of the transcription factor CEBPB (SEQ ID NO: 3) and expression thereof.

7. Method of any one of the previous claims, further comprising insertion of the coding sequence of the gene of the transcription factor RUNX1 (SEQ ID NO: 4) and expression thereof.

8. Method of any one of the previous claims, further comprising insertion of the coding sequence of the gene of the transcription factor IRF8 (SEQ ID NO: 5) and expression thereof.

9. Method of any one of the previous claims, further comprising insertion of the coding sequence of the gene of the transcription factor SALL1 (SEQ ID NO: 6) and expression thereof.

10. Method of any one of the previous claims, wherein the transcriptional regulator protein is the reverse tetracycline transactivator (rtTA) (SEQ ID NO: 20) and the activity thereof is controlled by doxycycline or tetracycline.

11. Method of any one of the previous claims, wherein the inducible promoter includes a Tet Responsive Element (TRE) (SEQ ID NO: 21).

12. Method of any one of the previous claims, wherein said first and said second genomic safe harbour sites are selected from the group consisting of the hROSA26 locus (SEQ ID NO: 22), the AAVS1 locus (SEQ ID NO: 23), the CLYBL gene (SEQ ID NO: 24), the CCR5 gene (SEQ ID NO. 25), the HPRT gene (SEQ ID NO. 26) or genes with the site ID 325 on chromosome 8 (SEQ ID NO: 27), site ID 227 on chromosome 1 (SEQ ID NO: 28), site ID 229 on chromosome 2 (SEQ ID NO: 29), site ID 255 on chromosome 5 (SEQ ID NO: 30), site ID 259 on chromosome 14 (SEQ ID NO: 31), site ID 263 on chromosome X (SEQ ID NO: 32), site ID 303 on chromosome 2 (SEQ ID NO: 33), site ID 231 on chromosome 4 (SEQ ID NO: 34), site ID 315 on chromosome 5 (SEQ ID NO: 35), site ID 307 on chromosome 16 (SEQ ID NO: 36), site ID 285 on chromosome 6 (SEQ ID NO: 37), site ID 233 on chromosome 6 (SEQ ID NO: 38), site ID 311 on chromosome 134 (SEQ ID NO: 39), site ID 301 on chromosome 7 (SEQ ID NO: 40), site ID 293 on chromosome 8 (SEQ ID NO: 41), site ID 319 on chromosome 11 (SEQ ID NO: 42), site ID 329 on chromosome 12 (SEQ ID NO: 43) and site ID 313 on chromosome X (SEQ ID NO: 44).

13. Method of any one of the previous claims, wherein said stem cell is a pluripotent stem cell, an induced pluripotent stem cell (iPSC), a neural progenitor cell, hematopoietic stem cell or an embryonic stem cell (ESC).

14. Method of any one of the previous claims, wherein said stem cell is a human or a mouse stem cell.

15. A microglia obtained by any one of the methods according to claims 1 to 14, preferably wherein the microglia expresses at least one microglia surface protein selected from the group consisting of ITGAM (CD11B) (SEQ ID NO: 45), ITGAX (CD11C) (SEQ ID NO: 46), CD14 (SEQ ID NO: 47), CD16 (SEQ ID NO: 48), ENTPD1 (CD39) (SEQ ID NO: 49), PTPRC (CD45) (SEQ ID NO: 50), CD68 (SEQ ID NO: 51), CSF1R (CD115) (SEQ ID NO: 52), CD163 (SEQ ID NO: 53), CX3CR1 (SEQ ID NO: 54), TREM2 (SEQ ID NO: 55), P2RY12 (SEQ ID NO: 56), TMEM119 (SEQ ID NO: 57), and HLA-DR (SEQ ID NO: 58).

16. Microglia according to claim 15 for use in therapy.

17. Use of microglia according to claim 15 or 16 for in vitro diagnostics of a disease.

18. Use of microglia according to claim 17, wherein the disease is selected from the group consisting of diseases of the central nervous system, preferably neurodegenerative diseases; more preferably Alzheimer's disease, Parkinson's disease, frontotemporal dementia or Amyotrophic Lateral Sclerosis; neuroinflammatory or autoimmune diseases, preferably Multiple Sclerosis, auto-antibody-mediated encephalitis or infectious diseases, neurovascular diseases; preferably stroke, vasculitis; traumatic brain injury, and cancer.

19. Use of microglia according to claim 15 or 16 for in vitro culturing with brain organoids.
